SEQUENCE LISTING (8 sequences)

SEQ ID NO: 1 - DNA, 20 bp, Artificial Sequence
  Synthetic primer
gggggacgatcgtcgggggg

SEQ ID NO: 2 - DNA, 20 bp, Artificial Sequence
  Synthetic primer
tgatcggaacttctggaggg

SEQ ID NO: 3 - DNA, 20 bp, Artificial Sequence
  Synthetic primer
tggcccagtacctgcttctc

SEQ ID NO: 4 - DNA, 18 bp, Artificial Sequence
  Synthetic primer
ccaggttgtctgcgactt

SEQ ID NO: 5 - DNA, 21 bp, Artificial Sequence
  Synthetic primer
cctgttgctgtagccgtattc

SEQ ID NO: 6 - DNA, 20 bp, Artificial Sequence
  Synthetic primer
ccagccgagccacatcgctc

SEQ ID NO: 7 - DNA, 21 bp, Artificial Sequence
  Synthetic primer
atgagccccagccttctccat

SEQ ID NO: 8 - protein, 9 aa, Artificial Sequence
  Synthetic peptide
SIINFEKEL

(19) United States
(12) Patent Application Publication
(10) Pub. No.: US 2011/0293658 A1
(43) Pub. Date: Dec. 1, 2011

(54) USE OF INKT OR TLR AGONISTS FOR PROTECTING AGAINST OR TREATING A DISEASE SUCH AS ACUTE INFECTION OR CANCER

(75) Inventors: Vincenzo Cerundolo, Oxford (GB); Carmela De Santo, Oxford (GB)

(73) Assignee: LUDWIG INSTITUTE FOR CANCER RESEARCH LTD., New York, NY (US)

(21) Appl. No.: 13/128,364

(22) PCT Filed: Nov. 12, 2009
(86) PCT No.: PCT/GB2009/051521

§ 371 Date: Aug. 3, 2011

(30) Foreign Application Priority Data
Nov. 12, 2008 (GB) 0820698.9

(51) Int. Cl. A61K 39/145; A61K 31/164; A61K 31/437; A61K 39/285; A61K 39/00; A61K 39/29; A61P 37/04; A61P 37/06; A61P 35/00; A61P 31/16; A61P 31/14; A61P 31/20; A61P 31/00; A61P 33/00; A61K 31/713

(52) U.S. Cl. 424/209.1; 424/184.1; 424/232.1; 424/265.1; 424/277.1; 424/228.1; 424/227.1

(57) ABSTRACT

A method of protecting a mammalian subject against, or treating, a disease, wherein the mammalian subject has elevated numbers and/or activities of MDSC comprising administering to the subject a pharmaceutically acceptable amount of an iNKT agonist, such as alpha galactosylceramide or an analogue thereof, or a TLR agonist, or a combination thereof.

### FIELD OF THE INVENTION

The invention relates to new prophylactic and therapeutic treatments involving the use of an iNKT agonist, such as α-GalCer or an analogue thereof, or a TLR agonist, or any combination thereof.

### BACKGROUND OF THE INVENTION

Adaptive immune responses may be compromised by a variety of immunosuppressive mechanisms by the expansion of CD40⁺ suppressive cells such as myeloid-derived suppressor cell (MDSC) numbers and activity. MDSC are comprised of immature dendritic cells, immature macrophages and granulocytes. MDSC, which express CD11b and Gr-1 markers, are capable of suppressing T cell proliferation and promoting tumor growth, due to the expression of both nitric oxide synthase 2 (NOS2) and arginase 1 (ARG1), resulting in the production of peroxynitrites under conditions of limited L-arginine availability. The use of selective inhibitors of NOS2 and ARG1 has confirmed the role of both enzymes in mediating MDSC suppressor activity and indicated that MDSC can modulate antigen specific immune responses during acute and chronic inflammatory conditions (De Santo et al, Proc Natl Acad Sci USA 102:4185-4190; Serafini et al, J Exp Med 203:2691-2702).

MDSC have been implicated in many conditions associated with immune suppression, including chronic microbial infection, severe trauma and many forms of cancer. For example, alteration of cytokines during polymicrobial sepsis (Delano et al, J Exp Med 204:1463-1474), parasitic infections (Mencacci et al, J Immunol 169:3180-3190), vaccinia virus infection (Bronte et al, Blood 96:3838-3846) and tumor development (e.g. Almand et al, J Immunol 166:678-689 Ochoa et al, Clin Canc Res, 13:721s-736s, Bronte et al, Nat Rev Immunol, 5:641-654, Yu et al, Nat rev Immunol, 7:41-51) causes a progressive accumulation of MDSC in the spleen, lymph nodes and bone marrow. In view of this apparently generalised immune suppression, it is thought that increased numbers or activities of MDSC may also limit vaccination efficiency and other forms of immunotherapy and increase susceptibility to infectious diseases.

Heithoff et al. (Infect Immun. 2008 November; 76(11):5191-9) report on the presence of MDSC in Salmonella infection but observe that a mutant form of Salmonella, the dam mutant, induces less MDSC than non-mutant forms and propose incorporation of such a mutant in their vaccine strain of Salmonella for greater efficacy.

In cancer, accumulation of myeloid-derived suppressor cells (MDSC) is an important mechanism of tumour immune evasion through suppression of anti-tumour immunity. Diaz-Montero C M, et al. (Cancer Immunol Immunother. 2009 January; 58(1):49-59) have shown that circulating MDSC levels correlate with clinical cancer stage and were significantly increased in cancer patients of all stages relative to healthy volunteers. Moreover, among stage 1V patients, those with extensive metastatic tumour burden had the highest percent and absolute number of MDSC. Hoechst B et al. (Gastroenterology. 2008 July; 135(1):234-43) propose that in hepatocellular carcinoma (HCC) MDSC exert their immunosuppressive function, by acting as tolerogenic antigen presenting cells capable of antigen uptake and presentation to tumour-specific regulatory T cells.

Effective abrogation of MDSCs or prevention of MDSC accumulation, in conjunction with immune activation therapy showed synergistic therapeutic effect when treating mice bearing large tumours (Serafini P et al. Cancer Res. 2008 Jul. 1; 68(13):5439-49 and Pan P Y, et al. Blood. 2008 Jan. 1; 111(1):219-28). Furthermore, in animal models of adoptive immunotherapy (AIT), anti-tumour efficacy of ex vivo-expanded antigen-specific T cells is compromised by the increased levels of myeloid-derived suppressor cells (MDSC). Combination of AIT with the depletion of MDSC, in vivo, results in the restoration of antigen-specific immunity and regression of antigen positive tumours (Morales J K et al. Cancer Immunol Immunother. 2008 Nov. 1).

Grizzle W E et al. (Mech Ageing Dev. 2007 November-December; 128(11-12):672-80) suggests that in mice the accumulation of MDSC contributes to the increase of tumour susceptibility as the age of mice increases. T cell cytotoxicity against implanted TS/A tumour cells exhibited a significant age-related decline in BXD12 mice which was correlated with the accumulation of MDSC in the spleen. Adoptive transfer of the accumulated MDSC from aged mice to 2-month-old BXD12 mice led to the delay of the rejection of implanted tumour cells and depletion of MDSC from aged BXD12 mice led to the slower growth of tumour.

Cachexia accompanies many chronic inflammatory diseases, including cancer. Lean tissue wasting is only one component of the cancer cachexia response, which also includes anaemia, anorexia, a hepatic acute phase protein response, and increased susceptibility to secondary infections. Recent evidence has focused on myeloid-derived suppressor cells (MDSC) that expands dramatically in the tumours and secondary lymphoid organs of animals with actively growing tumours. These MDSC are metabolically active and secrete large quantities of inflammatory cytokines and chemokines with the potential to produce cachexia (Winfield R D et al. JPEN J Parenter Enteral Nutr. 2008 November-December; 32(6):651-5).

Accordingly, it is an object of the invention to provide methods for reducing the number and/or activities of MDSC and/or in modulating MDSC activity and phenotype and in so doing enhance immune responses which would otherwise be diminished in the presence of immunosuppressive MDSCs.

Mechanisms that modulate the frequency and activity of MDSC in vivo remain ill defined. However, it has been shown that CD restricted NKT cells (type II NKT cells) can enhance MDSC suppressive activity by secreting IL-13 (Terabe et al, Cancer Res. 2006 Apr. 1; 66(7):3869-75).

Invariant NKT cells (iNKT) cells are a subset of lymphocytes recognizing endogenous and/or exogenous glycolipid antigens in the context of CD molecules. iNKT cells facilitate anti-microbial and anti-tumor responses by bridging the innate and adaptive immune systems. Although there is evidence that iNKT cells play an important role against viral infections, the mechanisms by which iNKT cells control viral infections remain unclear.

Previously, iNKT agonism has been achieved and studied through use of a glycolipid, α-Galactosylceramide (compound A or “αGal-Cer” hereafter). αGal-Cer and its derivatives, have been known as biologically active agents for some time and are currently being investigated for use in the treatment of various diseases, including cancer, infectious diseases, sepsis and allergy. See, e.g., U.S. Pat. No. 5,936,076 to Higa, et al., and U.S. Pat. No. 6,531,453 to Taniguchi, et al., describing several derivatives as anti-tumor agents as well as immunostimulators, both of these being incorporated by reference in their entirety. αGal-Cer has been developed as a potential therapeutic compound and taken into clinical testing, see, for example, Giaccone et al., Clin Canc Res, 8, 3702-3709 (2002).

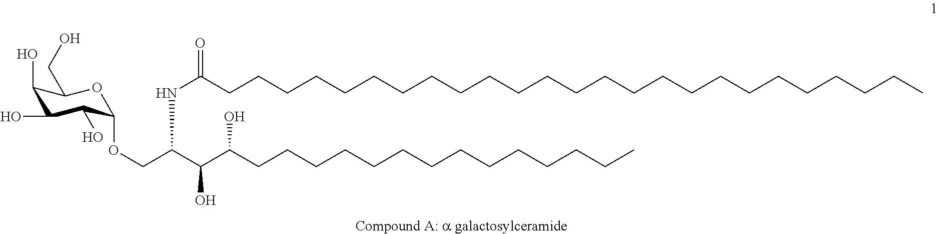

Singh, et al., J. Immunol., 163:2373 (1999), and Burdin, et al., Eur. J. Immunol., 29:2014 (1999), have shown that αGal-Cer and the major histocompatibility complex-class I like protein, CD1d, potentiate Th2Th1-mediated, adaptive immune responses, via activation of Val4 natural killer T cells (iNKT). α-GalCer/CD1d-dependent stimulation of iNKT cells results in the rapid production of Th1 and Th2 cytokines, such as IFN-γ and IL-4. Kawano et al Cancer Res. 1999 Oct. 15; 59(20):5102-5. Furthermore, glycosylceramides activate Val4 NKT cells through a CD1d-restricted, TCR-mediated mechanism Kawano, et al. Science. 1997 Nov. 28; 278(5343):1626-9. Furthermore, CD1d-restricted recognition of synthetic glycolipid antigens by human natural killer T cells demonstrates a striking parallel in the specificity of NK-T cells in humans and mice, Spada et al. J Exp Med 188, 1529-34 (1998). Through increased expression of the CD40 ligand (CD40L) early in the immune response, CD1-reactive T cells direct CD40L-dependent dendritic cell development and the initiation of adaptive immunity, Vincent M S et al. Nat Immunol. 3(12):1163-8 (2002). The linkage of innate to adaptive immunity via maturing dendritic cells in vivo requires CD40 ligation in addition to antigen presentation and CD80/86 costimulation, Fujii, et al. J Exp Med. 2004 Jun. 21; 199(12):1607-18 and the direct interaction of NKT cells with dendritic cells enhances CD4+ and CD8+ T cell responses to soluble antigen in vivo, Hermans, I. F. et al. J. Immunol. 171, 5140-5147 (2003).

Various publications describe synthesis of αGal-Cer and its derivatives. An exemplary, but by no means exhaustive list of such references includes Morita, et al., J. Med. Chem., 38:2176 (1995); Sakai, et al., J. Med. Chem., 38:1836 (1995); Morita, et al., Bioorg. Med. Chem. Lett., 5:699 (1995); Takakawa, et al., Tetrahedron, 54:3150 (1998); Sakai, et al., Org. Lett., 1:359 (1998); Figueroa-Perez, et al., Carbohydr. Res., 328:95 (2000); Plettenburg, et al., J. Org. Chem., 67:4559 (2002); Yang, et al., Angew. Chem., 116:3906 (2004); Yang, et al., Angew. Chem. Int. Ed., 43:3818 (2004); and, Yu, et al., Proc. Natl. Acad. Sci. USA, 102(9):3383-3388 (2005).

WO 2007/050668 which is incorporated in its entirety herein describes a group of analogues of αGal-Cer that substantially mimic the binding properties of α-GalCer with the human CD molecule, but differs significantly in the interaction with T-cell receptors (TCR), leading to unexpected and advantageous properties compared to α-GalCer. In particular, it describes compounds having the formula:

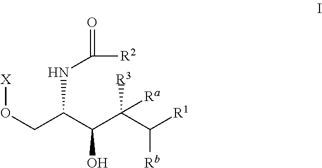

in which
R¹ represents a hydrophobic moiety adapted to occupy the F′ channel of human CD1d,
R² represents a hydrophobic moiety adapted to occupy the A′ channel of human CD1d, such that R′ fills at least at least 30% of the occupied volume of the F′ channel compared to the volume occupied by the terminal nC₁₄H₂₉ of the sphingosine chain of α-galactosylceramide when bound to human CD1d and R² fills at least 30% of the occupied volume of the A′ channel compared to the volume occupied by the terminal nC₂₅H₅₁ of the acyl chain of α-galactosylceramide when bound to human CD1d
R³ represents hydrogen or OH,
R^{a} and R^{b} each represent hydrogen and in addition, when R³ represents hydrogen, R^{a} and R^{b} together may form a single bond,
X represents or —CHA(CHOH)ₙY or —P(═O)(O⁻)OCH₂(CHOH)ₘY, in which Y represents CHB₁B₂,
n represents an integer from 1 to 4, m represents 0 or 1,
A represents hydrogen,
one of B₁ and B₂ represents H, OH or phenyl, and the other represents hydrogen or one of B₁ and B₂ represents hydroxyl and the other represents phenyl,
in addition, when n represents 4, then A together with one of B₁ and B₂ together forms a single bond and the other of B₁ and B₂ represents H, OH or OSO₃H,
and pharmaceutically acceptable salts thereof.

The antigen-binding site of mouse and human CD1d (hCD1d) molecules is composed of two channels: A′ and F′ channels in mouse CD1d, which connect directly to the surface. For consistency with the mouse CD1d literature, the phytosphingosine chain-binding channel in hCD1d, which is referred to as the C′ channel by Koch et al. Nat. Immunol. 6:819-826 (2005), is here referred to as the F′ channel, see McCarthy et al. J Exp Med. 204(5): 1131-1144 (2007).

WO 2008/128207 which is incorporated in its entirety herein relates to synthetic αGal-Cer analogues, and their use in immunotherapy. In particular, it describes compounds having the formula:

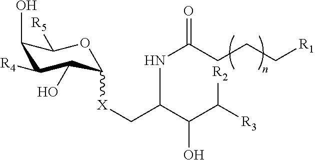

wherein, n is 0 to 25; X is selected from O and S; R₁ is selected from H, CH₃, and phenyl, where phenyl is optionally substituted with H, OH, OCH₃, F, CF₃, phenyl, phenyl-F, C₁-C₆ alkyl, or C₂-C₆ branched alkyl; R₂ is selected from OH and H; R₃ is selected from C₁-C₁₅ alkyl, and phenyl, where phenyl is optionally substituted with H, OH, OCH₃, F, CF₃, phenyl, C₁-C₆ alkyl, or C₂-C₆ branched alkyl; R₄ is selected from OH, OSO₃H, OSO₃Na, and OSO₃K; and R₅ is selected from CH₂OH and CO₂H; or a pharmaceutically acceptable salt thereof.

Toll-like receptor (TLR) agonists are currently being investigated for use in the treatment of various diseases, including cancer, infectious diseases, sepsis and allergy. TLR agonists are molecules (small molecules, pathogen derived lipids, proteins, DNA, RNA) targeting one or more of the toll-like receptors including TLR3, TLR4, TLR5, TLR7, TLR8 and TLR9. TLR agonists are being developed for use in the preparation of pharmaceutical compositions to interfere, to modulate and to regulate responses of the innate and adaptive immune system. Those include enhancement of immune responses (including vaccination) and modulation of immune responses. Such use of TLR agonists to enhance the immune response includes induction of immunological memory, cytotoxic T cells, cytokine release and augmentation of innate immunity (phagocytosis, cytokine release and cytolytic function). In particular, TLR agonists can be used as an adjuvant for T- and B-cell vaccination. Enhancement further includes induction of reactivity of immune cells to viral, bacterial, parasitic antigens and against weak or tumour antigens. The use of TLR agonists to break tolerance in anergic T and B cells, e.g. against tumour antigens, is also being considered. Furthermore, TLR agonists are used as adjuvants in vaccination against tumour-defined antigens and immunostimulatory substances in an ongoing immune response against tumours.

Hermans et al (J Immunol. 2007 Mar. 1:178(5):2721-9) which is incorporated in its entirety herein confirm that the actions of iNKT agonists and TLR agonists can be complementary and synergistic and WO 2007/050668 proposes such combinations. Salio et al (Proc Natl Acad Sci USA. 2007 Dec. 18; 104(51):20490-5.) which is incorporated in its entirety herein indicate an additional mechanistic rationale for such iNKT agonist/TLR agonist combinations. WO 2009/133378 which is incorporated in its entirety herein by reference proposes combinations of iNKT agonists and TLR agonists in particulate formulations including liposomes. Specifically, it relates to products which comprise (i) a support and (ii) a B cell receptor (BCR)-binding antigen attached to the support, and optionally (iii) an immunostimulant (which may comprise an iNKT agonist or a TLR agonist) attached to the support.

An object of the invention is to provide new prophylactic and therapeutic treatments involving the use of iNKT agonists, such as α-GalCer and its analogues, and of TLR agonists either alone or in combination.

### SUMMARY OF THE INVENTION

Accordingly, a first aspect of the invention provides a method of protecting a mammalian subject against, or treating, a disease, such as an infectious disease or cancer, wherein the mammalian subject has elevated numbers and/or activities of MDSC comprising administering to the subject a pharmaceutically acceptable amount of an iNKT agonist, such as α-GalCer or an analogue thereof, or a TLR agonist, or any combination thereof. The method is particularly useful for treating diseased subjects in which levels of MDSC are increased through their disease state or protecting or treating the elderly and subjects exposed to extreme levels of stress in whom MDSC levels have increased in such as manner as to increase their susceptibility to disease and/or impair their ability to recover from disease.

According to a second aspect the invention provides a method of protecting a mammalian subject against, or treating, a disease, in which elevated numbers and/or activities of MDSC occur, or are induced by the disease state, comprising administering to the subject a pharmaceutically acceptable amount of an iNKT agonist, such as α-GalCer or an analogue thereof, or a TLR agonist, or any combination thereof.

The disease may be an acute infection which induces elevated numbers and/or activities of MDSC, such as influenza A virus (IAV), pandemic flu, vaccinia virus infection or polymicrobial sepsis, or a chronic infection which induces elevated numbers and/or activities of MDSC, such as chronic microbial infection, severe trauma, parasitic infections or cancer.

According to a further aspect the invention provides a method of stimulating an immune response in a mammalian subject against a disease such as an infectious disease or cancer comprising administering to the subject (i) a pharmaceutically acceptable amount of an iNKT agonist, such as α-GalCer or an analogue thereof, or a TLR agonist, or any combination thereof, and subsequently administering to the subject (ii) an immunotherapeutic composition such as a vaccine against the disease. Advantageously, this method of immunization is more efficacious than current methods in that improved humoral immune responses (such as higher antibody titres) and/or cellular immune responses (such as T cell responses) are observed and/or lower doses are required. Preferably the immunotherapeutic composition of (ii) is administered a sufficient time after the treatment of (i) for MDSC levels to be diminished and to allow protective immune responses to be achieved, for example preferably about 1 to 28 days or more preferably about 1 to 15 days.

According to a further aspect the invention provides a method of protecting a mammalian subject against, or treating, an acute infectious disease which induces a high level of MDSCs in the infected subject comprising administering to the subject a pharmaceutically acceptable amount of an iNKT agonist, such as α-GalCer or an analogue thereof, or a TLR agonist, or any combination thereof. The disease may be, for example, influenza A virus (IAV), pandemic flu, vaccinia virus infection or polymicrobial sepsis. The method is particularly useful as a prophylactic treatment which may be used prior to and to help prevent an expected disease outbreak or pandemic. Preferably administration is before disease exposure as part of a vaccine formulation incorporating other components of such a vaccine including antigens or immunogens. Advantageously, this method renders subjects less susceptible to the disease and/or enables a more rapid recovery if they are subsequently infected.

According to a further aspect the invention provides a method of prophylactically treating a mammalian subject at risk of exposure to an infectious disease which causes elevated numbers and/or activities of MDSC comprising administering to the subject a pharmaceutically acceptable amount of an iNKT agonist, such as α-GalCer or an analogue thereof, or a TLR agonist, or any combination thereof. The method is particularly useful as a prophylactic treatment to help prevent an expected infection when the subject is about to be exposed to a known risk of disease exposure such as may occur in the outbreak of a pandemic or in a hospital based nosocomial infection. The disease may be an acute infection which induces elevated numbers and/or activities of MDSC, such as influenza A virus (IAV), pandemic flu, vaccinia virus infection or polymicrobial sepsis, or a chronic infection which induces elevated numbers and/or activities of MDSC, such as chronic microbial infection, or a parasitic infection, or a nosocomial infection, such as hepatitis B virus, hepatitis C virus or pandemic flu.

Preferably the α-GalCer analogue is selected from those described in WO 2007/050668, or any combination thereof. More preferably, the α-GalCer analogue is threitolceramide:

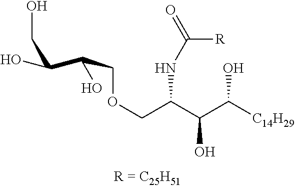

Preferably the TLR agonist is selected from poly I:C (TLR3), MPL (TLR4), imiquimod (TLR7), R848 (TLR7/8), R852 (TLR7), R853 (TLR8), R34240 (TLR7), R854 (TLR7/8), CpG (TLR9), or any combination thereof.

Preferred combinations of iNKT agonists and TLR agonists are described in WO 2009/133378, which is incorporated herein in its entirety, and may be formulated in liposomes.

### DETAILED DESCRIPTION OF THE INVENTION

The invention is based upon a novel immunomodulatory role of iNKT cells which could be harnessed to inhibit the immunosuppressive activity of MDSC. Specifically, compounds which stimulate iNKT cells may be used to suppress the numbers and/or activity of MDSC and/or to in modulate MDSC activity and phenotype. In doing so, we are able to enhance an immune response. Accordingly, the invention provides new prophylactic and therapeutic vaccine formulations and methods of treatment using these formulations.

It is demonstrated herein that acute influenza A virus (IAV) infection in mammals results in substantial elevations in MDSC numbers and immunosuppressive activity and that treatment of both mouse and human MDSC cell populations from patients with IAV infection with iNKT agonism or TLR agonism can diminish levels of MDSC and thus provide for novel prophylactic and therapeutic modalities. Additionally similar effects are shown in MDSC population from melanoma patients with relevance for therapeutic vaccine approaches in both melanoma and other forms of cancer in which MDSC are prevalent and limit immune responses.

In accordance with the invention we provide a method of protecting a mammalian subject against, or treating, a disease, in which elevated numbers and/or activities of MDSC occur, or are induced by the disease comprising administering to the subject a pharmaceutically acceptable amount of an iNKT agonist, such as α-GalCer or an analogue thereof, or a TLR agonist, or any combination thereof, which may be formulated within a micro- or nano-particle such as but not limited to a liposome formulation. The method will diminish MDSC levels and allow subsequent immune recovery. Immune responses may be monitored until disease clearance or amelioration is achieved. Retreatment may be administered as required.

The invention also provides a method of protecting a mammalian subject against, or treating, a disease, wherein the mammalian subject has elevated numbers and/or activities of MDSC comprising administering to the subject a pharmaceutically acceptable amount of an iNKT agonist, such as α-GalCer or an analogue thereof, or a TLR agonist, or any combination thereof.

The methods of the invention are particularly useful for treating diseased subjects in which levels of MDSC are increased through their disease state or protecting or treating the elderly and subjects exposed to extreme levels of stress, such as personnel involved in military combat, or persons suffering from diseases such as cancer. In such subjects, MDSC levels have increased in such a manner as to increase their susceptibility to disease and/or impair their ability to recover from disease. The frequency of MDSC may be defined by CD11b expression and optionally additionally by CD15 expression. Thus, in healthy volunteers, the frequency of MDSC, as defined by CD11b expression, ranges between 10-30% and these cells do not have high suppressive activity. In contrast, in cancer patients, for example melanoma patients, and in patients with infectious disease such as influenza-infected individuals, we observed frequencies as high as 90% of CD11b+ cells out of the total peripheral blood monocytes. Similarly, in healthy volunteers, the frequency of MDSC, as defined by CD11bCD15 expression, ranges between 10-30% and these cells do not have high suppressive activity. In contrast, in cancer patients, for example melanoma patients, and in patients with infectious disease such as influenza-infected individuals, we observed frequencies as high as 90% of CD11bCD15+ cells out of the total peripheral blood monocytes. Thus, a mammalian subject having elevated numbers and/or activities of MDSC may be considered as one having greater than 25% CD11b+ cells out of the total peripheral blood monocytes, more preferably greater than 30%, 35%, 40%, 50%, 60%, 70%, 80% or 90% CD11b+ cells out of the total peripheral blood monocytes. Additionally or alternatively, a mammalian subject having elevated numbers and/or activities of MDSC may be considered as one having greater than 25% CD11bCD15+ cells out of the total peripheral blood monocytes, more preferably greater than 30%, 35%, 40%, 50%, 60%, 70%, 80% or 90% CD11bCD15+ cells out of the total peripheral blood monocytes. Such elevation of MDSC may also be associated with an up-regulation of CD15 expression, for example CD15 expression may be 2-fold higher than in a healthy subject, preferably 10-fold higher, 20-fold higher, 50-fold higher or most preferably 100-fold higher than in a healthy subject.

Elderly or older subjects are known to have elevated numbers and/or activities of MDSC and are more likely to suffer from infectious diseases and develop diseases such as cancer. The probability of developing cancer is 8.58% (1 in 12) for males and 8.97% (1 in 11) for females between the ages 40 to 59 and increases to 16.25% (1 in 6) for males and 10.36% (1 in 10) for females between the ages 60 to 69. Above age 70 the probability of developing cancer increases to 38.96% (1 in 3) for males and 26.31% (1 in 4) for females. Elderly persons are considered to be those aged greater than 60, preferably aged greater than 65, more preferably aged greater than 70, greater than 80 or greater than 90.

Also provided is a method of treatment which involves an initial course of treatment to diminish MDSC levels (with an iNKT agonist or a TLR agonist or a combination of both) followed by immunisation or other form of immunotherapy which would be less effective in the presence of MDSC e.g. therapeutic antibody therapy, with any immunogenic composition.

Thus, we provide a method of stimulating an immune response in a mammalian subject against a disease such as an infectious disease or cancer comprising administering to the subject (i) a pharmaceutically acceptable amount of an iNKT agonist, such as α-GalCer or an analogue thereof, or a TLR agonist, or any combination thereof, and subsequently administering to the subject (ii) an immunotherapeutic composition such as a vaccine against the disease. The vaccine may be a prophylactic or therapeutic vaccine against an infectious disease or a cancer vaccine. The immunotherapeutic composition may be an immunotherapy such as ex vivo stimulated autologous immune cells or T cells against a disease such as cancer.

There is substantial preclinical experimental support for the concept that antitumor antibodies in their roles as opsonins can promote the immunogenicity of both human and murine tumor antigens. Immunization of mice with dendritic cells pulsed with antibody-opsonized tumor antigens acquired via either FcgR-mediated endocytosis (Dhodapkar et al, J Exp Med 2002; 195:125-33., Kalergis et al, J Exp Med 2002; 195:1653-9.) or phagocytosis (Akiyama et al, J Immunol 2003; 170:1641-8.) induces CD4- and CD8-mediated tumor immunity. Cross-presentation mediated by FcgRs on human dendritic cells has been shown to enhance the presentation of multiple myeloma antigens to patient-derived T-cells, (Dhodapkar et al, J Exp Med 2002; 195:125-33) suggesting that uptake of antibody-opsonized tumor cells and cellular fragments by antigen-presenting cells could lead to antigenic/epitope spreading and the induction of immunity to several tumor-associated antigens.

Additionally, recent clinical studies have shown a positive correlation between the presence of FcgR polymorphisms with favourably higher affinities for IgG and improved clinical outcomes in rituximab-treated patients (Cartron et al, Blood 2002; 99:754-8. Weng et al, J Clin Oncol 2003; 21:3940-7, Treon et al, J Clin Oncol 2005; 23:474-81, Kim et al, Blood 2006; 108:2720-5). These studies have confirmed preclinical observations and established that Fc-FcgR interactions are critical to rituximab's antitumor antibody clinical efficacy.

An association between the same polymorphic alleles and clinical outcomes has also been reported for the chimeric antibody targeting the epidermal growth factor receptor, cetuximab, in patients with colorectal cancer (Zhang et al, J Clin Oncol 2007, 25:3712-3718). Additionally, (Musolino et al, J Clin Oncol. 2008 Apr. 10; 26(11):1789-96) demonstrated a similar association between FcgR polymorphisms and clinical outcome in patients with HER-2-amplified breast cancer receiving trastuzumab plus taxane.

Importantly, Taylor et al, (Clin Cancer Res 2007; 13(17) Sep. 1, 2007), demonstrated humoral and cellular responses in patients receiving transtuzumab passive immunotherapy consistent with the induction of active immune responses by immune effector functions triggered by, for example, ADCC (antibody dependent cellular cytotoxicity), data consistent with the concept of active immunization in a post-passive immunotherapy setting.

Since such effects would be expected to be at least partially mediated by cross-presentation mechanism induced by, for example, dendritic cell uptake of tumor cells initiated by opsonisation or phagocytosis of trastuzumab bound tumor cells, enhancing such cross-presentation pathways in the context of the profoundly immunosuppressed microenvironment within a tumor is expected to yield enhanced immune responses. Hence, the current inventors, having previously demonstrated with others eg Silk et al (J Clin Invest, 2004, December 114(12) 1800-1811) the ability of iNKT cell activation to facilitate cross-presentation, claim that it is particularly useful to condition or pre-treat patients due to be given an active immunization or passive immunotherapy with a pretreatment using iNKT agonists and/or TLR agonists or combinations thereof designed to inhibit or diminish the immunosuppressive effects of MDSC before subsequent initiation of an immunotherapy protocol. The optimum dosing schedule between such pretreatment and subsequent active immunization with a vaccine is dictated by the dosing schedule which achieves an optimum modulation of MDSC populations consistent with enhancing subsequent immune effects and is expected to be easily established by those skilled in the art by monitoring of MDSC levels during pretreatment with iNKT agonists and/or TLR agonists or combinations thereof.

Teng et al, (Canc Res, 2007, 67:15, 7495-504) demonstrate in a tumor model in mice that vaccination in the absence of exogenously administered tumor antigen but with the appropriate immunostimulatory signals can achieve efficacy and induce regression of tumor. Immunization protocols which do not require addition of exogenous antigen are thus also contemplated by the current invention. Thus, treatment of tumors with an iNKT agonist and/or a TLR agonist or a combination of both thereof modulates MDSC levels and allows the immunostimulatory activity of the iNKT agonist and/or a TLR agonist or a combination of both thereof either from the same administration of from subsequent administrations whilst MDSC levels remain suppressed to induce cross-presentation of endogenous tumor antigen with a resulting immune response and tumor regression.

Other forms of immunotherapy which can be combined with a conditioning or pretreatment with an iNKT agonist and/or TLR agonist or combination of both to inhibit or modulate MDSC levels include cellular therapy such as T-cell or NKT-cell therapy with the data included here clearly supporting the relevance of such therapy with NKT cells.

Passive immunotherapies where there is clear evidence of ADCC effects in man, including but not limited to the use of rituximab (anti-CD20), trastuzumab (anti-HER2), cetuximab (anti-EGFR) are each candidates for pretreatment with an iNKT agonist, such as α-GalCer or an analogue thereof, or a TLR agonist, or any combination thereof to reduce MDSC levels and enhance subsequent efficacy of immune effects which utilise in whole or in part cross-presentation mediated by antigen presenting cells such as dendritic cells.

Although the above literature references describing prior art in the field of assessing the potential contribution of cross-presentation relate to cancer, the discovery by the current inventors of the presence of MDSC populations in an acute viral infection (IAV) demonstrate that similar approaches of pretreatment to modulate MDSC levels followed by active vaccination using vaccines against IAV credible.

Additionally, since an active infection by, for example, IAV provides large amounts of pathogen antigen, pre-exposure treatment of a patient who is about to be exposed to IAV or immediate post exposure treatment of a patient who has been exposed to IAV antigens through infection with an iNKT agonist and/or a TLR agonist or a combination of both thereof to modulate MDSC levels on infection and facilitate cross-presentation of pathogen antigens is expected to prove a useful therapy (in this setting the provision of pathogen antigen via infection obviates the need for additional antigen to be provided exogenously in the form of a vaccine).

Additional situations in which a prophylactic course of treatment with an iNKT agonist and/or a TLR agonist or a combination of both thereof is expected to be useful include exposure with an infectious pathogen in which exposure can be anticipated to allow optimum prophylaxis such as elective surgery in a hospital in-patient setting where exposure to nosocomial infections is anticipated. Thus prophylactic administration of an iNKT agonist and/or a TLR agonist or a combination of both thereof is expected to be useful in, for example, infection with Staphylococcus, (including MRSA), Enterococcus, E coli, C difficile, C albicans and other nosocomial infections.

Additional, post exposure situations in which treatment before the development of symptomatic disease with an iNKT agonist and/or a TLR agonist or a combination of both will be useful include exposure to blood borne diseases through, for example surgical exposure such as HBV, HIV, and HCV, and pandemic flu infections to healthcare workers. Rabies may also represent a post exposure, pre-disease symptomatic disease situation where treatment as proposed in the current invention will be useful.

In addition to many forms of cancer including melanoma, B cell lymphoma, non-small cell lung cancer, breast cancer, colon cancer, pancreatic cancer, prostate cancer, glioblastoma, renal cell carcinoma and influenza A virus (IAV), such diseases include infectious diseases such as described earlier, and in particular influenza A virus (IAV), vaccinia virus infection, Salmonella infection, chronic microbial infection, severe trauma, polymicrobial sepsis, parasitic and helminth infections and many forms of cancer including melanoma, B cell lymphoma, non-small cell lung cancer, breast cancer, colon cancer, pancreatic cancer, prostate cancer, glioblastoma, renal cell carcinoma.

Preferably the treatment would be a single or repeat administration of iNKT agonist or TLR agonist or a combination in an active dose, optionally followed by monitoring of MDSC levels to confirm a diminished level, at which point a vaccine or immunotherapy such as a therapeutic antibody or cellular therapy would be administered at the accepted dose level and frequency for that therapy. For example, the time between administration of iNKT agonist or TLR agonist or combination thereof and administration of vaccine or immunotherapy or the like may be from 1 day to 28 days, preferably from 3 days to 14 days, most preferably from 5 days to 10 days. Preferably, repeat administration of an iNKT agonist or TLR agonist or combination thereof would not be given more frequently than once weekly.

The method of treatment may also involve treatment of any disease in which MDSCs are elevated which is prophylactic in settings where the risk of subsequent pathogen exposure is high e.g. nosocomial infections (e.g. where subjects are involved in elective surgery, the precise time of potential exposure may be anticipated), health workers known to have been exposed to infection e.g. blood borne exposure such as HBV, HCV, pandemic flu etc. For example, a person exposed to pandemic flu but who is not yet in the most acute phase of infection may be immediately treated to activate iNKT cells in preparation for an elevation in MDSCs. MDSCs numbers and/or activities would be diminished by prior iNKT cell activation.

Thus, we provide a method of protecting a mammalian subject against, or treating, an acute infectious disease which induces a high level of MDSCs in the infected subject comprising administering to the subject a pharmaceutically acceptable amount of an iNKT agonist, such as α-GalCer or an analogue thereof, or a TLR agonist, or any combination thereof. The disease may be an acute infectious disease such as influenza A virus (IAV), vaccinia virus infection, chronic microbial infection or a parasitic infections. This method may be particularly useful as a prophylactic treatment which may be used prior to and/or to help prevent an expected disease outbreak or pandemic. Preferably administration is before disease exposure as part of a vaccine formulation incorporating other components of such a vaccine including antigens or immunogens. Advantageously, this method renders subjects less susceptible to the disease and/or enables a more rapid recovery if they are subsequently infected.

Also provided is a method of prophylactically treating a mammalian subject at risk of exposure to an infectious disease which causes elevated numbers and/or activities of MDSC comprising administering to the subject a pharmaceutically acceptable amount of an iNKT agonist, such as α-GalCer or an analogue thereof, or a TLR agonist, or any combination thereof. This method may be useful to help prevent an expected infection when the subject is about to be exposed to a known risk of disease exposure such as may occur in the outbreak of a pandemic or in a hospital based nosocomial infection. The disease may be an acute infection which induces elevated numbers and/or activities of MDSC, such as influenza A virus (IAV), pandemic flu, vaccinia virus infection or polymicrobial sepsis, or a chronic infection which induces elevated numbers and/or activities of MDSC, such as chronic microbial infection, or a parasitic infection, or a nosocomial infection, such as hepatitis B virus, hepatitis C virus or pandemic flu.

In accordance with the invention, it is particularly useful to induce immune responses through immunization/vaccination against or to treat diseases such as but not limited to, cancer and infectious diseases, in which elevated numbers and/or activities of MDSC occur or are induced by the disease state.

Such diseases include infectious diseases such as described earlier and in particular influenza A virus (IAV), vaccinia virus infection, Salmonella infection, chronic microbial infection, severe trauma, polymicrobial sepsis, parasitic and helminth infections and many forms of cancer including melanoma, B cell lymphoma, non-small cell lung cancer, pancreatic cancer, prostate cancer, glioblastoma, renal cell carcinoma. Additional disease states include but are not limited to cachexia, inflammatory bowel disease and eczema.

The antigens may be derived from bacteria, mycobacteria, viruses, fungi and parasites. It is to be understood that antigens derived from a particular microorganism can be used to prevent and/or treat an infection of that microorganism. Below is provided a list of microorganisms from which antigens may be derived.

Bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacilliis moniliformis, Treponema pallidium, Treponema pertenue, Leptospira, Rickettsia, and Actinomyces israelli.

Viruses include, but are not limited to, interoviruses (including, but not limited to, viruses that the family picornaviridae, such as polio virus, coxsackie virus, echo virus), rotaviruses, adenovirus, hepatitus. Specific examples of viruses that have been found in humans include but are not limited to Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Viruses that infect both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-I, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in vertebrate animals include, but are not limited to, members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1. Sendai virus, Hemadsorption virus, Parainifluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, ONyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family Paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that infect vertebrate animals include but are not limited to the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immuno-compromised or immunosuppressed subjects include, but are not limited to, candida albicans (an organism which is normally part of the respiratory tract flora), cryptococcus neoformans (sometimes in normal flora of respiratory tract), and various aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Fungi include but are not limited to microsporum or traicophyton species, i.e., microsporum canis, microsporum gypsum, tricofitin rubrum, Cryptococcus neoformans, Histoplasma capsulatum, Coccidioides immitis, Blaslomyces dermatitidis, Chlamydia trachomatis, and Candida albicans.

Parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni.

Influenza A virus is an infectious disease of birds and mammals caused by RNA viruses of the family Orthomyxoviridae. Wild aquatic birds are the natural hosts for a large variety of influenza A, but viruses are transmitted to other species and may then cause devastating outbreaks in domestic poultry or give rise to human influenza pandemics. The type A viruses are the most virulent human pathogens among the three influenza types and cause the most severe disease. The influenza A virus can be subdivided into different serotypes based on the antibody response to these viruses. The serotypes that have been confirmed in humans are H1N1, which caused Spanish flu in 1918; H2N2, which caused Asian Flu in 1957; H3N2, which caused Hong Kong Flu in 1968; H5N1, a pandemic threat in the 2007-08 flu season; H7N7, which has unusual zoonotic potential; H1N2, endemic in humans and pigs; and other serotypes including H9N2; H7N2; H7N3 and H10N7.

Cancers which may be mentioned include Basal Cell Carcinoma, Breast Cancer, Leukemia, Burkitt's Lymphoma, Colon Cancer, Esophageal Cancer, Bladder Cancer, Gastric Cancer, Glioblastoma, Head and Neck Cancer, Hepatocellular Cancer, Hodgkin's Lymphoma, Hairy Cell Leukemia, Wilms' Tumor, Thyroid Cancer, Thymoma and Thymic Carcinoma, Testicular Cancer, T-Cell Lymphoma, Pancreatic Cancer, Prostate Cancer, Non-Small Cell Lung Cancer, Small Cell Lung Cancer, Liver Cancer, Renal Cell Cancer, and Melanoma.

Infectious disease means a disorder caused by one or more species of bacteria, viruses, fungi, parasites or protozoans, referred to as pathogens. In this invention, pathogens are exemplified, but not limited to, Mycobacterium tuberculosis, M. leprae, Pseudomonas aeruginosa, Shigella dysenteria, Salmonella typhi, S. paratyphi, Staphylococcus aureus, Streptococcus hemolyticus, Hemophilus pneumoniae, Escherichia coli serotype 0157, Chlamydia species, Helicobacter species; human immunodeficiency viruses HIV-1, -2, and -3, human herpes virus (HSV-I and -II), non-A non-B non-C hepatitis virus, human papilloma virus (HPV), cytomegalovirus (CMV), human T-cell leukemia virus (HTLV-I and II), feline leukemia virus (FeLV), simian immune deficiency virus (SIV), and rous sarcoma virus (RSV), pox viruses, rabies viruses; Aspergillus species; Entamoeba histolytica, Giardia species; and Newcastle disease virus.

Viral infections that may be mentioned include Viral Hepatitis for example HBV, HCV; Herpes virus infection for example Herpes simplex virus, other skin tropic viruses like human papiloma virus, Lung tropic viruses like influenza virus or respiratory syncytial virus, chronic or acute viral infections with HIV, EBV or CMV or combinations of viral infections or viral and bacterial infections.

Bacterial infections include bacterial infections of the lung, for example Haemophilus influenzae, or mycobacteria, for example Mycobacterium tuberculosis, and bacterial infections of the gut, for example helicobacter pylori, or the skin, like Staphylococcus aureus.

Compounds suitable for use in accordance with any of the methods of the invention are α-GalCer or analogues of α-GalCer or TLR agonists. Analogues of α-GalCer include those described in WO 2007/050668 and WO 2008/128207, the entireties of which are incorporated herein by reference. Particularly preferred are the compounds of WO 2007/050668, in particular compound 3: threitolceramide:

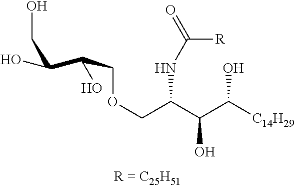

Also preferred are is compound 2: glycerolceramide of WO 2007/050668

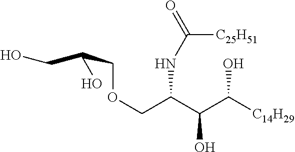

Preferred combinations of iNKT agonists and TLR agonists are described in WO 2009/133378.

Any Toll-like receptor (TLR) agonist may be used in accordance with the invention. An exemplary, but by no means exhaustive list of such TLR agonists includes poly I:C (TLR3), MPL (TLR4), imiquimod (TLR7), R848 (TLR7/8), R852 (TLR7), R853 (TLR8), R34240 (TLR7), R854 (TLR7/8), or CpG (TLR9).

Compositions containing conjugated forms of an iNKT agonist such as α-GalCer or an analogue thereof or a TLR agonist or a combination thereof may be used in accordance with the invention. Examples of conjugated compositions and the use of such are described in WO07051004A2 and WO 2009/133378, both of which are incorporated herein in their entirety. Compositions containing mixtures of two or more iNKT agonists, such as α-GalCer or analogues thereof, or TLR agonists, may be used in accordance with the invention. A preferred composition contains α-GalCer or an analogue thereof or in combination with a TLR agonist. The combination of iNKT agonist and TLR agonist may be advantageous as there two classes of compounds synergise in their combined ability to activate iNKT cells (WO 2007/050668 and Salio et al, PNAS 2007). These compositions could be used as liposome formulations as described in WO2006002642, which is incorporated herein in its entirety.

These compounds may be used in admixture with a pharmaceutically acceptable excipient, carrier or adjuvant. Such formulations are generally well known to the person skilled in the art and may be analogous to those described in EP 0 609 437B, EP-A-1 437 358 and WO 2004/028475, the contents of which are herein incorporated by reference.

The compounds may also be used individually within a micro- or nano-particle such as but limited to a liposome formulation i.e. as a lipid formulation of an iNKT agonist alone or as a TLR agonist alone or as part of a formulation incorporating both an iNKT agonist and a TLR agonist in a combination liposome.

Compositions of the invention may be administered via intravenous, intradermal, intramuscular, intranasal or subcutaneous routes of administration.

Compositions in a form suitable for topical administration to the lung include aerosols, e.g. pressurised or non-pressurised powder compositions; compositions in a form suitable for oesophageal administration include tablets, capsules and dragees; compositions in a form suitable for administration to the skin include creams, e.g. oil-in-water emulsions or water-in-oil emulsions; compositions in a form suitable for administration intravenously include injections and infusions; and compositions in a form suitable for administration to the eye include drops and ointments.

The pharmaceutical composition may comprise preferably less than 80% and more preferably less than 50% by weight of, α-GalCer or an analogue of α-GalCer or a TLR agonist or combinations thereof, in admixture with a pharmaceutically acceptable diluent or carrier. Examples of such diluents and carriers are:
for tablets and dragees—lactose, starch, talc, stearic acid;
for capsules—tartaric acid or lactose; and
for injectable solutions—water, alcohols, glycerin, vegetable oils, or liposomes.

Preferred compositions may be formulated in liposomes, for examples such combinations of iNKT agonists and TLR agonists are described in WO 2009/133378.

When the composition is to be administered to the lung it may be inhaled as a powder which may be pressurised or non-pressurised. Pressurised powder compositions may contain a liquefied gas propellant or a compressed gas. In non-pressurised powder compositions the active ingredient in finely divided form may be used in admixture with a larger-sized pharmaceutically acceptable carrier comprising particles of up to, for example, 100 μm in diameter.

Suitable inert carriers include, e.g. crystalline lactose.

For the above mentioned uses the doses administered will, of course, vary with compound employed, the mode of administration and the treatment desired. However, in general, satisfactory results are obtained when α-GalCer or an analogue of α-GalCer or a TLR agonist or a combination thereof is administered at a daily dosage of from about 1 μg to about 20 mg per kg of animal body weight, preferably given in divided doses 1 to 4 times a day or in sustained release form. Preferably the dose for a typical 70 kg person is approximately 3.5 mg.

For man the total daily dose is in the range of from 20 μg to 1,400 mg and unit dosage forms suitable for administration comprise from 20 μg to 1,400 mg of the compound admixed with a solid or liquid pharmaceutical diluent or carrier. A preferred daily dose is in the range of from 50 μg to 5000 μg. Such a dose may be administered on 3 times over a 4 week period, for example on days 1, 8 and 15 of a 4-weekly cycle.

Preferably the treatment would be a single or repeat administration of iNKT agonist or

TLR agonist or a combination in an active dose, optionally followed by monitoring of MDSC levels to confirm a diminished level. Preferably, repeat administration of an iNKT agonist or TLR agonist or combination thereof would not be given more frequently than once weekly.

When the treatment to diminish MDSCs is prior to a subsequent treatment (for example vaccination), the subsequent treatment should preferably be administered at a time when the initial treatment has had the effect of lowering the number and/or activity of MDSC. Preferably the time between treatments is from 1 day to 28 days, more preferably from 1 to 14 days, from 1 to 10 days or from 1 and 5 days.

Additionally, when the treatment to diminish MDSCs is prior to a subsequent treatment (for example vaccination), the subsequent treatment may be accompanied by further treatment with the iNKT agonist or TLR agonist or combination thereof. This will (a) provide ongoing inhibition of MDSCs as well as (b) active immunisation using the iNKT agonist or TLR agonist (and endogenous antigen).

### EXAMPLES

The examples below refer to the appended figures. Some of the figures contain colour and originals of these figures are available from the applicant upon request.

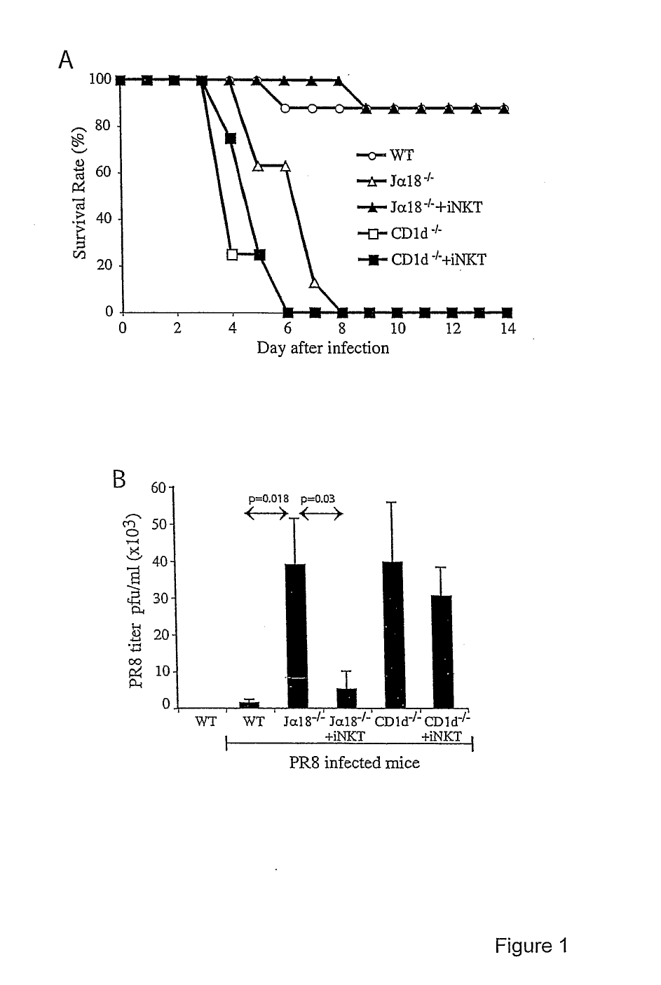

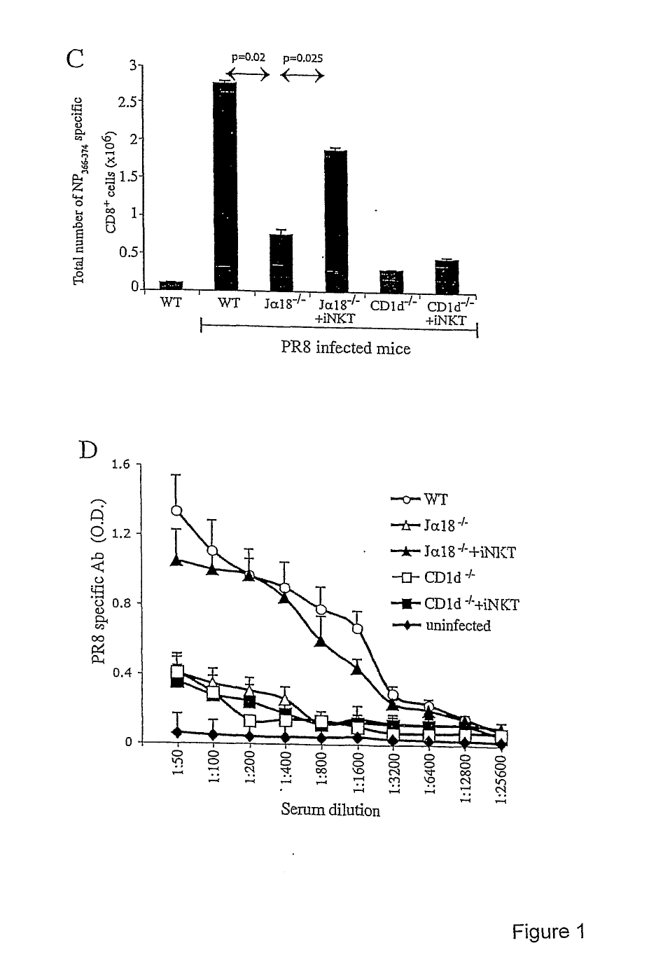

FIG. 1. Adoptive transfer of iNKT cells mediates protection from lethal doses of PR8.

WT, Jα18^{-/-} and CD1d^{-/-} mice (n=8/group) were injected i.n. with PR8 (3×10⁴ pfu/mouse). Liver purified iNKT cells were adoptively transferred i.v. into Jα18^{-/-} (Jα18^{-/-}+iNKT) and CD1d^{-/-} (CD1d^{-/-}+iNKT) mice 1 day post infection.

(A) Increased mortality of Jα18^{-/-} mice following PR8 infection is prevented by the adoptive transfer of iNKT cells. Survival rate is shown as the percentage of live mice at different time points after the infection. Data are representative of 5 separate experiments.

(B) PR8 titer in Jα18^{-/-} mice is reduced by the adoptive transfer of iNKT cells. Lung homogenates from PR8 infected mice were assayed for number of plaque forming units (pfu) 6 days post infection. Statistical analyses, performed using Student's T test, are shown.

(C) Total number of NP₃₆₅₋₃₇₄ specific CTL in PR8 infected Jα18^{-/-} mice is restored by the adoptive transfer of iNKT cells. The number of NP₃₆₅₋₃₇₄ specific CTL represents the average of results obtained in n=8 mice/group (±SD). Statistical analyses, performed using Student's T test, are shown.

(D) Anti PR8 antibody titers in PR8 infected Jα18^{-/-} mice are restored by the adoptive transfer of iNKT cells. The titer of anti-PR8 specific IgG was measured at day 6 in the sera of mice infected with PR8 (3×10⁴ pfu).

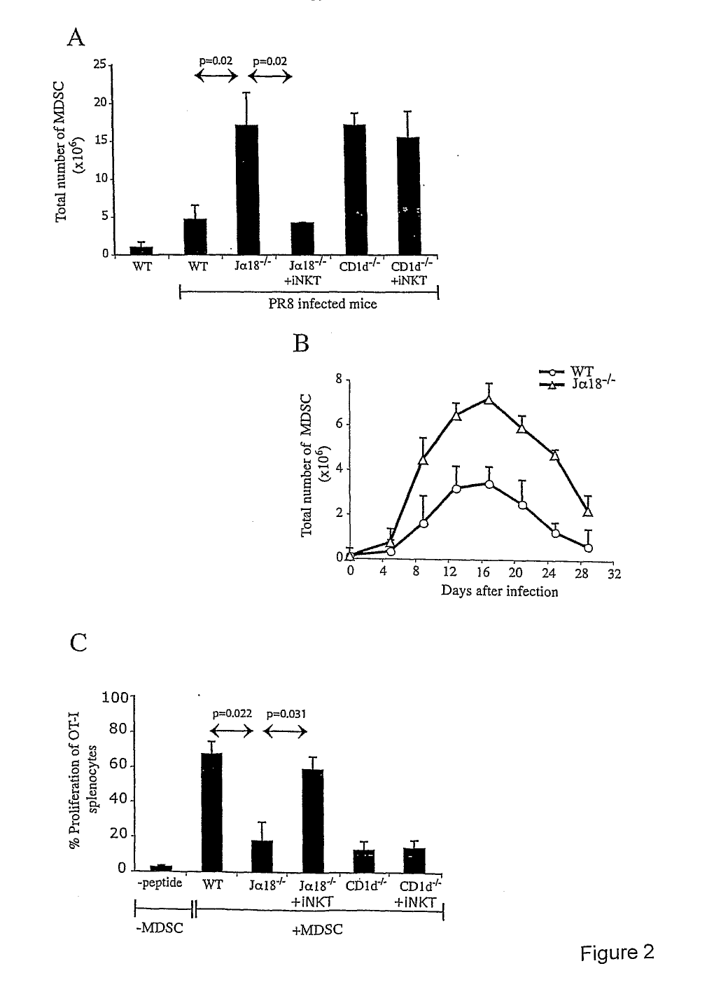

FIG. 2. Adoptive transfer of iNKT cells reduces PR8 induced MDSC expansion.

(A) Adoptive transfer of iNKT cells into PR8 infected Jα18^{-/-} mice reduced the total number of lung MDSC in infected Jα18^{-/-} mice. WT, Jα18^{-/-} and CD1d^{-/-} mice were injected i.n. with PR8 (3×10⁴ pfu/mouse) and liver purified iNKT cells were adoptively transferred i.v. 24 h later. Lungs were digested with collagenase (as described in Methods) as soon as the acute symptoms occurred ˜(5-6 days post infection). MDSC expansion was analysed staining lung homogenates with CD11b and GR-1 antibodies. Data represents the average of n=5 mice/group ±S.D. Statistical analyses, performed using Student's T test, are shown.

(B) Injection of sub-lethal doses of PR8 (3×10² pfu) reduces the total numbers of lung infiltrating MDSC, which peaked at approximately day 20 after the infection. Data represents the average of n=5 mice/group±S.D.

(C) Adoptive transfer of iNKT cells reduces in vitro suppressive activity of PR8 induced MDSC. MDSC were purified from lung homogenates of PR8 infected mice (n=5 mice per group) using Gr-1-coated magnetic beads. CFSE labelled OT-I proliferation was analysed in the presence of lung derived MDSC from PR8 infected mice (n=5 mice per group). The data are expressed as described in the Methods. As negative control, proliferation of unpulsed CFSE labelled OT-I splenocytes in the absence of MDSC is shown. 85% of added CFSE labelled OT-I splenocytes proliferated in the absence of MDSC and in the presence of the SIINFEKL peptide (100% proliferation). Statistical analyses, performed using Student's T test, are shown.

(D) The suppressive activity of MDSC from PR8 infected mice is reduced by treating with anti CD40 agonist antibody and NOS2 and ARG1 inhibitors. CFSE labelled OT-I proliferation was analysed in the presence of lung derived MDSC of infected mice (n=5 mice per group). Lung purified MDSC were left untreated (▪) or were treated in vitro with either anti-CD40 agonist antibody (□) or with L-NMMA and NOHA ( [symbol] ) and then added to OT-I splenocytes. As negative control, proliferation of unpulsed OT-I splenocytes in the absence of MDSC is shown ( [symbol] ). The addition of L-NMMA and NOHA to OT-I splenocytes in the absence of MDSC did not affect OT-I proliferation (data not shown). The data are expressed as described in the Methods. 98% of added CFSE labelled OT-I splenocytes proliferated in the absence of MDSC and in the presence of the SIINFEKL peptide (100% proliferation). Statistical analyses, performed using Student's T test, are shown.

(E) Adoptive transfer of MDSC from infected mice suppresses the expansion of vaccine driven UTY₂₄₆₋₂₅₄ specific CD8⁺ T cell responses. Naïve female WT mice were injected i.v. with DC from male mice and one day later, were injected i.v. with MDSC from shown PR8 infected mice (WT, Jα18^{-/-} and CD1^{-/-}) which received or not liver purified iNKT. A week later, mice were boosted with UV-inactivated vaccinia-UTY₂₄₆₋₂₅₄ minigene. UTY₂₄₆₋₂₅₄ CTL responses were assessed by ex-vivo tetramer staining, 7 days after vaccinia boosting. Data represents the average of n=5 mice per group.

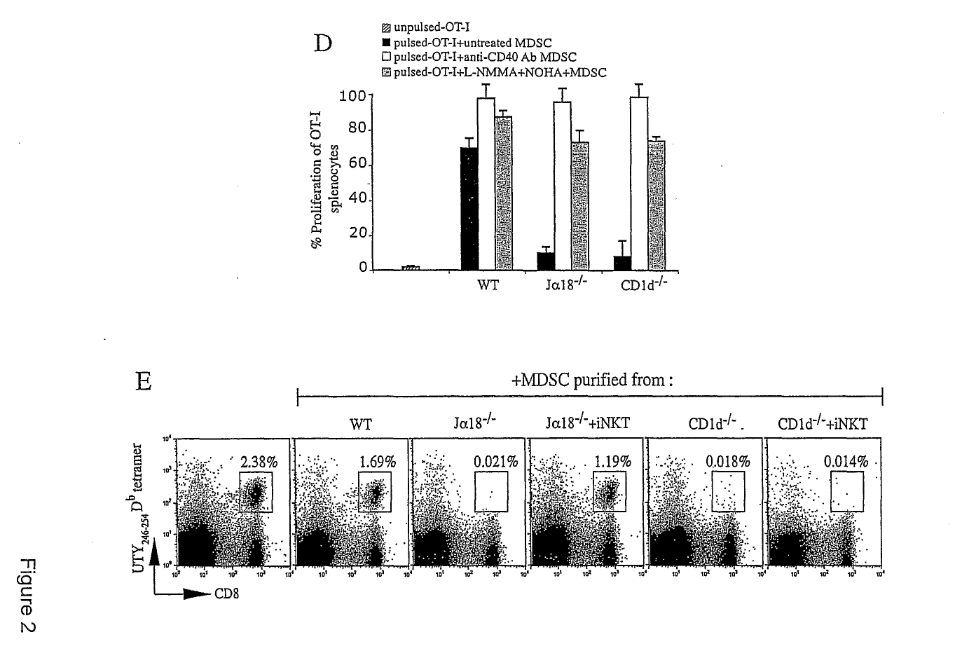

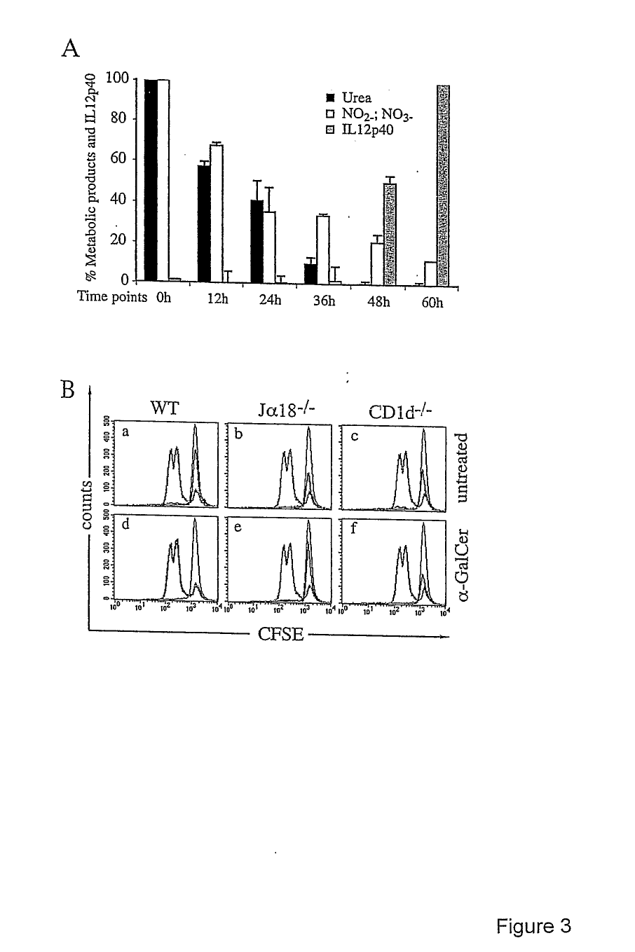

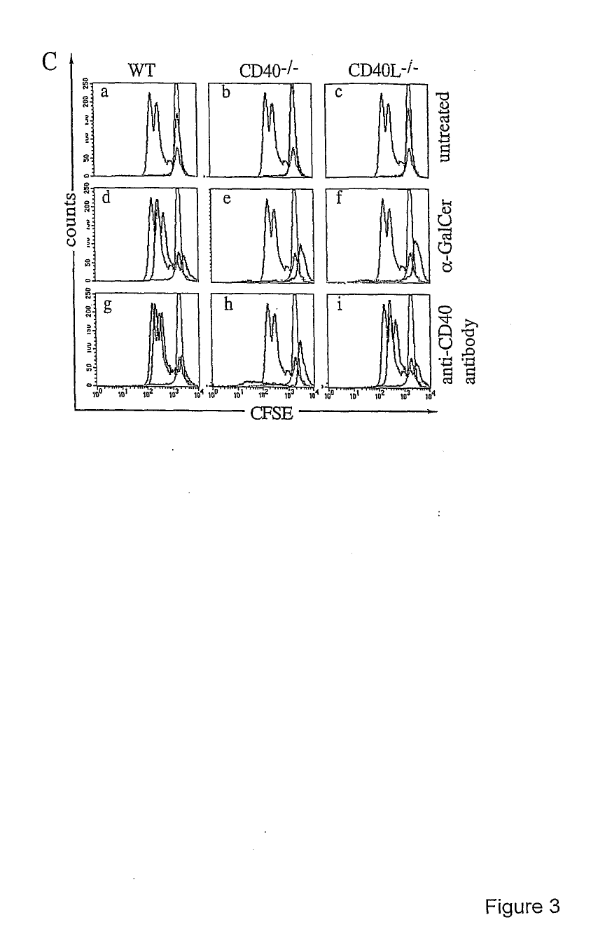

FIG. 3. The cross-talk between iNKT and MDSC is CD1d and CD40 dependent.

(A) Loss of ARG1 and NOS2 activity in α-GalCer pulsed MDSC incubated with BM derived iNKT cells. MDSC were treated with α-GalCer in the presence of iNKT cells and collected at different time points. ARG1 and NOS2 activities were measured using a colorimetric assay to evaluate urea and nitrate/nitrite release, respectively (De Santo et al. 2005. Proc Natl Acad Sci USA 102:4185-4190. Serafini et al. 2006. J Exp Med 203:2691-2702). An ELISA was used to evaluate IL-12p40 production. Values are shown as a percentage of time point 0. The maximum amount of urea released by untreated MDSC (1×10⁶) was 91.7 μg. The maximum amount of nitrate (NO₂) and nitrite (NO₃) measured in the supernatant of untreated MDSC was 120 μM. The maximum amount of IL-12p40 was 200 ng/ml.

(B) Incubating α-GalCer pulsed MDSC with BM derived iNKT cells abolishes their suppressive function. CFSE labelled OT-I proliferation was analysed in the presence (red) or absence (green) of MDSC derived from WT, Jα18^{-/-} or CD1d^{-/-} mice. MDSC were left untreated or pulsed with α-GalCer. Proliferation of OT-I cells without SIINFEKL peptide (black) is superimposed in all panels.

(C) The effect of iNKT cells on α-GalCer pulsed MDSC is CD40 dependent. CFSE labelled OT-I proliferation was analysed in the presence (red) or absence (green) of MDSC derived from WT, CD40^{-/-} or CD40L^{-/-} mice. MDSC were left untreated or pulsed with α-GalCer. To assess the role of the CD40 molecules, BM-derived MDSC were treated with agonist anti-CD40 antibody for 48 h. Proliferation of CFSE labelled OT-I cells without SIINFEKL peptide (black) is superimposed in all panels.

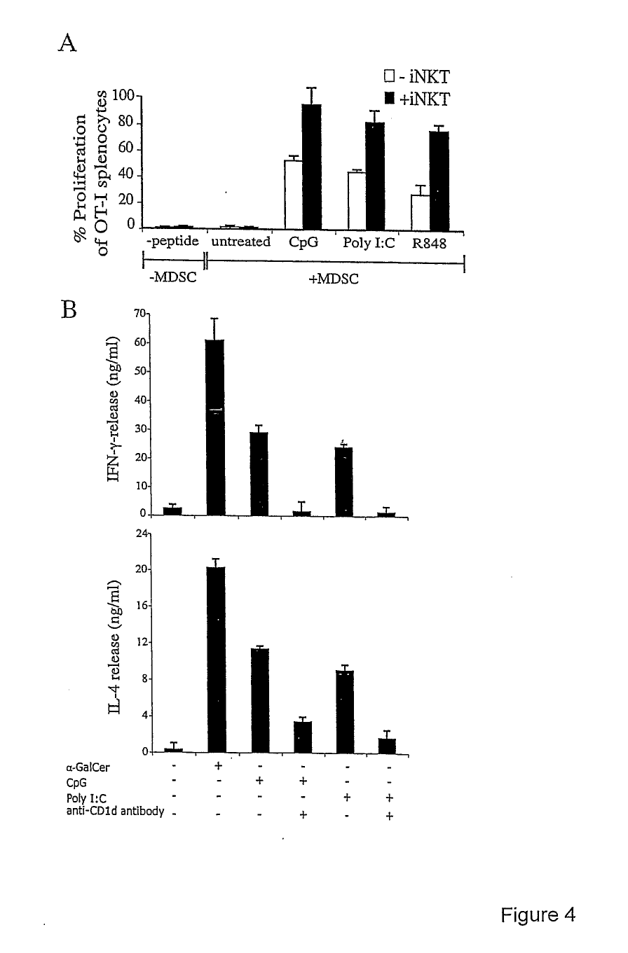

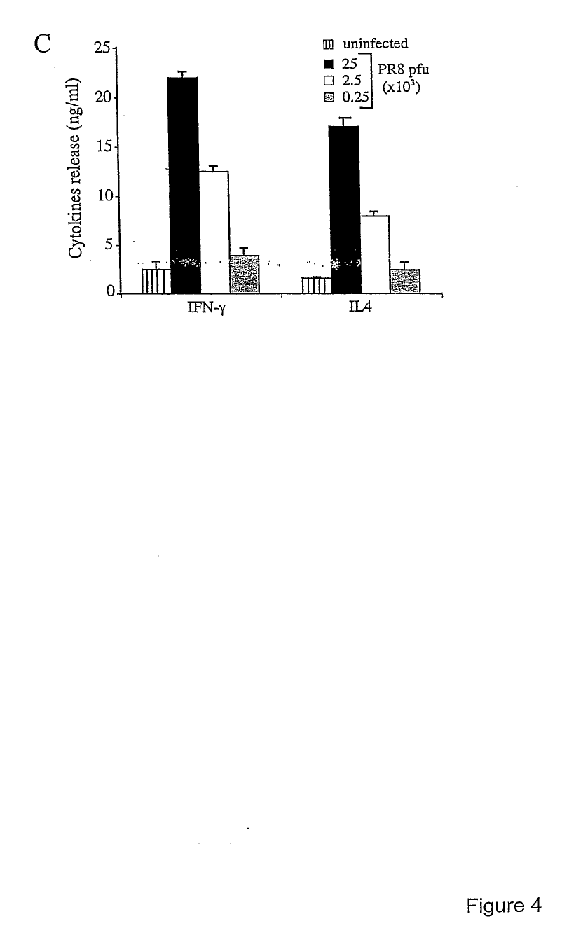

FIG. 4. Infection of MDSC with PR8 fosters their ability to activate iNKT cells.

(A) TLR agonists rescue suppressive activity of MDSC. BM derived MDSC were treated with TLR agonists in the presence or absence of liver purified iNKT as described in Methods CFSE labelled OT-I proliferation was analysed in the presence of TLR-agonist treated MDSC in the presence (▪) or absence (□) of iNKT cells. Data represent the average of 3 replicates ±S.D and are representative of 3 separate experiments. As negative control, proliferation of unpulsed CFSE labelled OT-I splenocytes in the absence of MDSC is shown. MDSC were incubated for 48 hr with shown TLR ligands.

(B) IFN-γ and IL-4 release from iNKT cells incubated for 48 h with CpG or Poly I:C treated MDSC, in the presence or absence of anti CD1d blocking antibody. Data represent the average of 3 replicates ±S.D and are representative of 3 separate experiments.

(C) Amounts of IFN-γ and IL-4 released by iNKT cells incubated in the presence of MDSC infected with increasing doses of PR8.

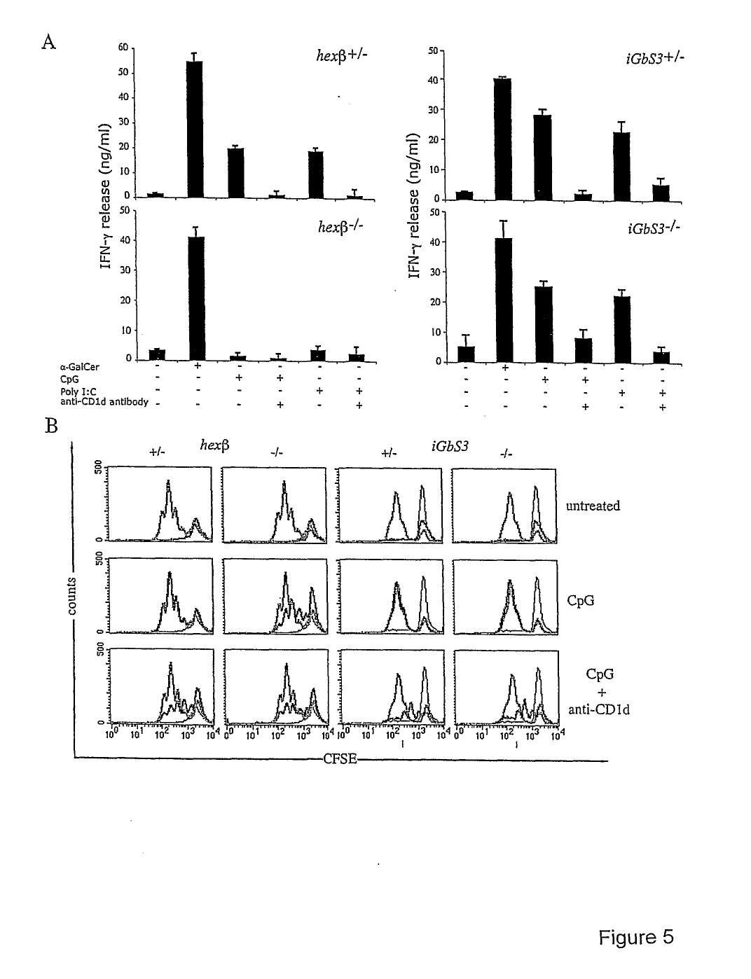

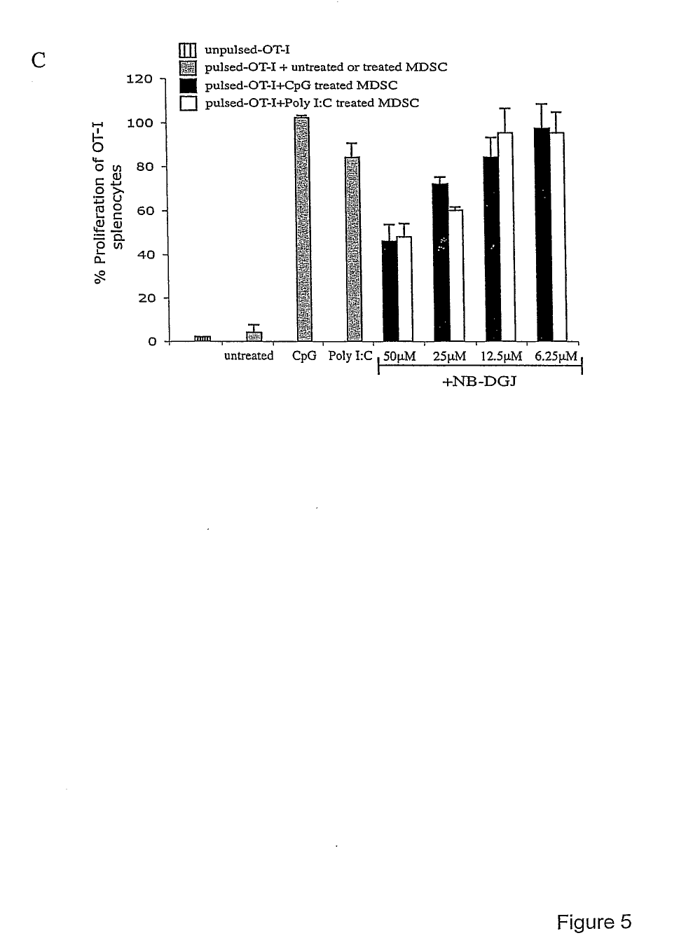

FIG. 5. TLR-L treated MDSC derived from hexβ^{-/-} mice fail to stimulate iNKT cells.

(A) iNKT cells derived from WT mice fail to produce IFN-γ when incubated with TLR-L matured MDSC derived from hexβ^{-/-} mice. Analysis of IFN-γ secretion by iNKT cells, derived from WT mice, co-incubated with TLR-L treated MDSC derived from either hexβ^{-/-} or iGb3S^{-/-} mice. To assess the role of CD1d molecules expressed on the surface of MDSC, TLR-L treated MDSC were also incubated with blocking anti-CD antibody.

(B) CpG treated hexβ^{-/-} MDSC fail to foster the cross-talk between iNKT cells and MDSC. The effect on CFSE labelled OT-I proliferation of TLR-L treated MDSC derived from either hexβ^{-/-} or iGb3S^{-/-} is shown. BM derived hexβ^{-/-} and iGb3S^{-/-} MDSC were treated with CpG in the presence or absence of blocking anti-CD1d antibody. Suppressive activity of MDSC was assessed 2 days later by analysing CFSE labelled OT-1 proliferation. Proliferation of OT-I cells in the presence (red) or absence (green) of MDSC is shown. Proliferation without the SIINFEKL peptide (black) is superimposed in all panels.

(C) Inhibition of GSL biosynthesis reduces iNKT cell activation by TLR-L-matured MDSC. MDSC derived from WT mice were incubated with either Poly I:C or CpG and then treated with increasing concentrations of NB-DGJ. MDSC were incubated with iNKT cells for 24 h and tested for their effect on OT-I proliferation. The addition of NB-DGJ to OT-I splenocytes in absence of MDSC did not affect OT-I proliferation (data not shown). The data are expressed as described in the Methods. As negative control, proliferation of peptide unpulsed CFSE labelled OT-I splenocytes in the absence of MDSC is shown ( [symbol] ). Treatment of MDSC with TLR-L ( [symbol] ) relieves CFSE labelled OT-I proliferation, as compared to the lack of OT-I proliferation observed after adding untreated MDSC. In contrast, combined treatment of MDSC with either CpG (▪) or Poly I:C (□) plus increasing doses of NB-DGJ reduces OT-I proliferation.

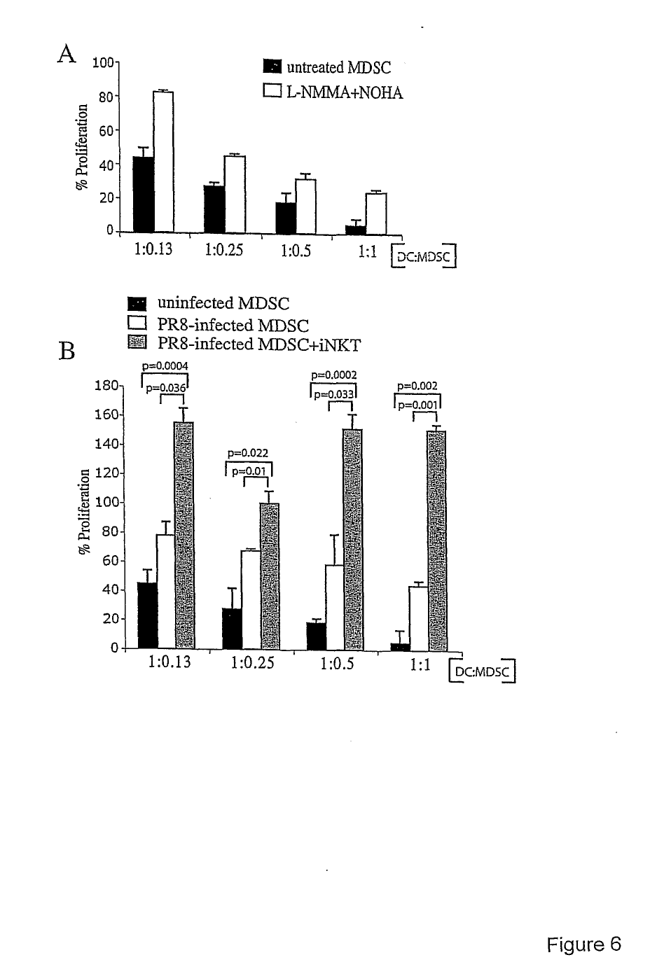

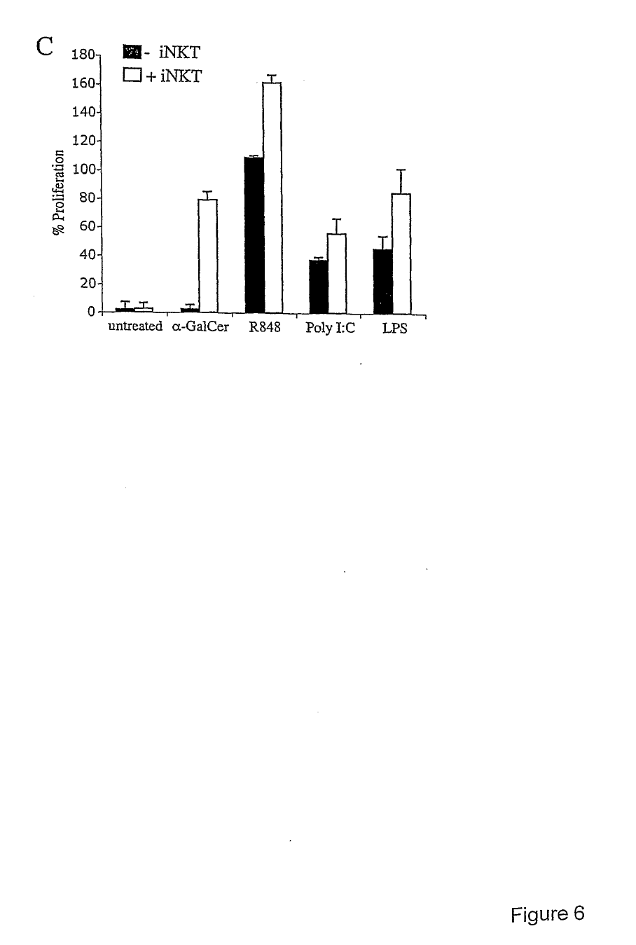

FIG. 6. Inhibition of alloreactive T cell proliferation by human MDSC can be rescued by iNKT cells.

(A) Healthy donors GM-CSF treated MDSC have MLR suppressive activity, which can be blocked by the addition of NOHA and L-NMMA. The data are expressed as described in the Methods. Addition of NOHA and L-NMMA to the MLR in the absence of MDSC does not affect PBL proliferation (data not shown), The ratio of DC to human GM-CSF treated MDSC is shown.

(B) PR8 infection of healthy donors' GM-CSF treated MDSC rescues MLR proliferation. Human GM-CSF treated MDSC were left untreated (▪) or infected with PR8 and co-cultured in the presence ( [symbol] ) or in the absence (□) of iNKT cells (2.5×10⁴) for 24 h. After irradiation, PR8 infected and uninfected MDSC were added to the MLR. The data are expressed as described in the Methods. Ratio of DC to human MDSC used is shown. Statistical analyses, performed using Student's T test, are shown.

(C) TLR-L incubation of healthy donors' GM-CSF treated MDSC rescues T cell proliferation. Human GM-CSF treated MDSC were treated with LPS (10 μg/ml), R848 (5 μg/ml), Poly I:C (10 μg/ml) or α-GalCer (100 ng/ml) and co-cultured in the presence (□) or absence (▪) of iNKT cells for 24 h. Treated cells were then washed and irradiated before adding them to the MLR. The data are expressed as described in the Methods. The ratio of DC to human GM-CSF treated MDSC used was 1:1.

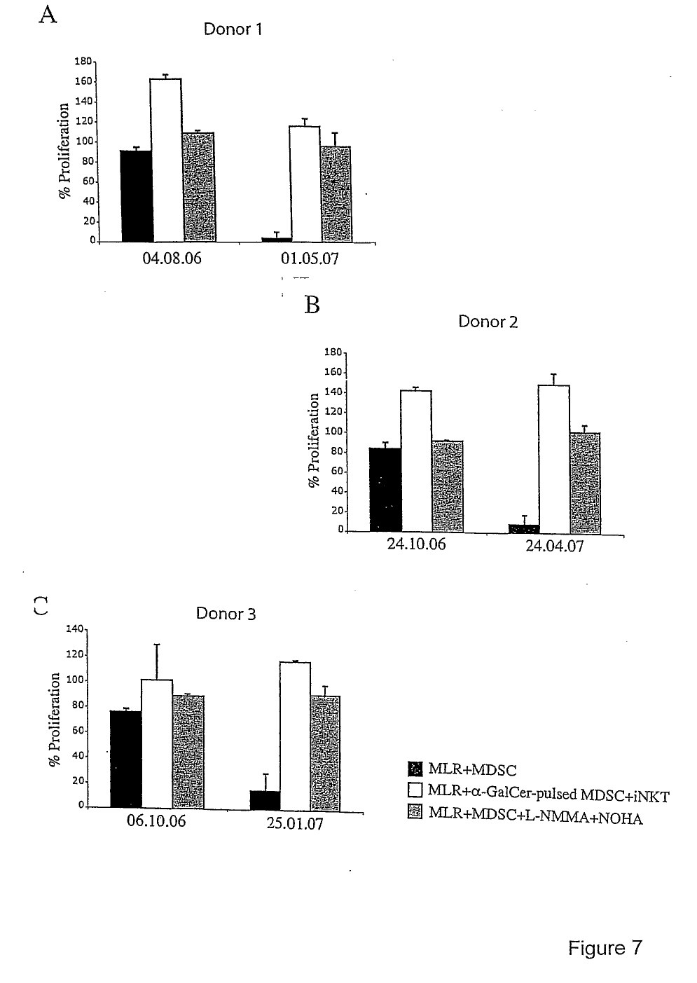

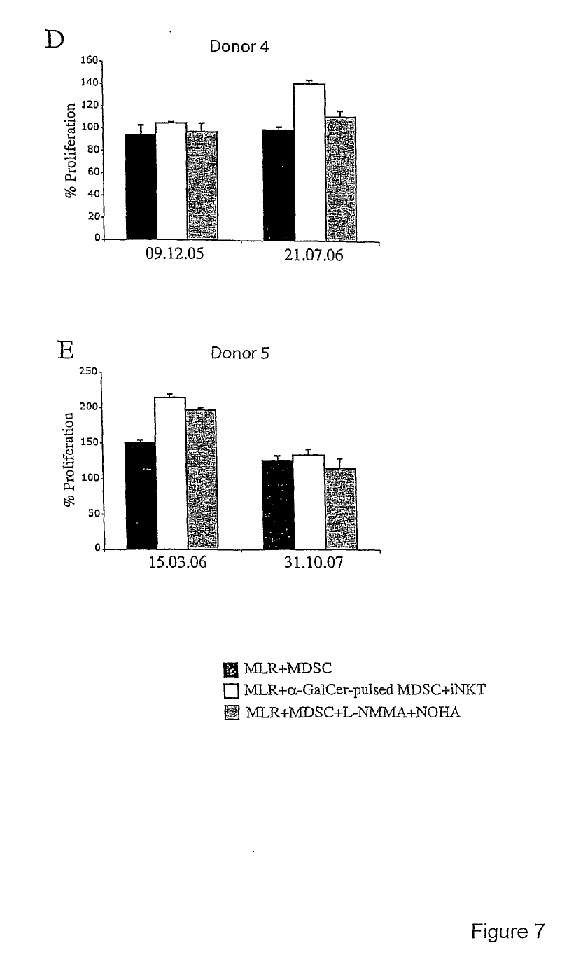

FIG. 7. Inhibition of T cell proliferation by MDSC purified from IAV-infected individuals. CD11b⁺ cells were bead purified from PBL derived from either IAV infected individuals (panels A, B, C) or from healthy donors (panels D and E). All donors were bled twice. Donors 1, 2 and 3: the first blood sample was collected before the clinical symptom onset, while the second blood sample was collected within 30-60 days after the acute respiratory illness. Donors 4 and 5 did not have any respiratory illness within the time frame of the collection of the two blood samples. Irradiated purified CD11b+ cells were then added to allogenic PBL and incubated with allogeneic irradiated DC. Purified CD11b⁺ cells were either untreated (▪), or treated with either α-GalCer (100 ng/ml) in presence of iNKT at a MDSC:iNKT ratio of 1:0.25 (□) or L-NMMA and NOHA ( [symbol] ). The data are expressed as described in the Methods. Addition of either iNKT cells or L-NMMA and NOHA to the alloreactive PBL in the absence of CD11b⁺ cells did not affect T cell proliferation (data not shown). The ratio of irradiated DC to purified irradiated CD11b⁺ cells was 1:1.

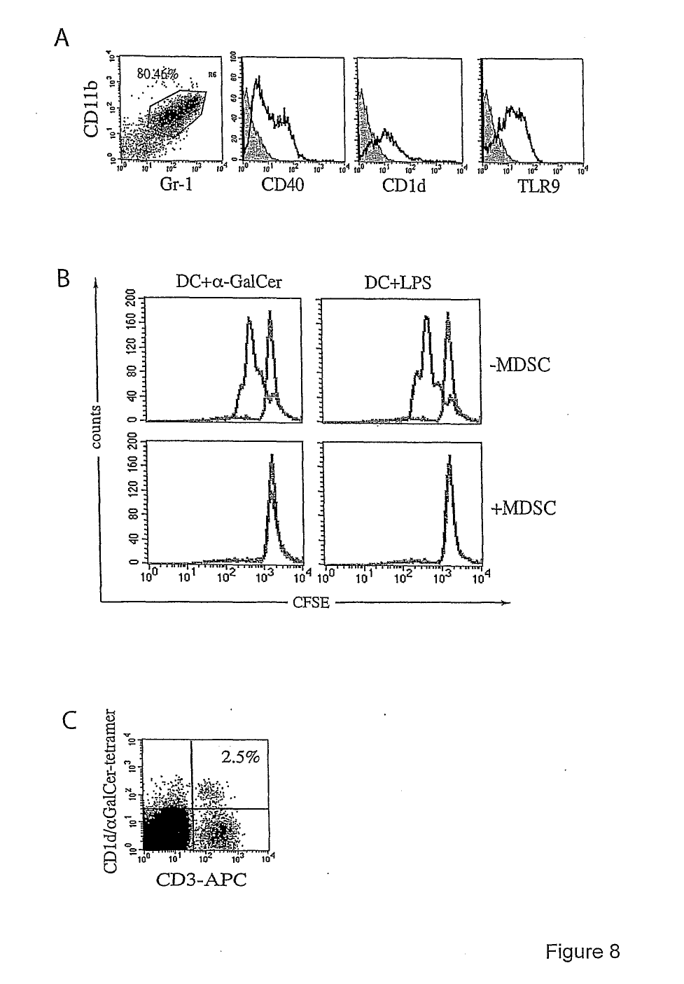

FIG. 8. Phenotypic and functional analysis of MDSC.

(A) Phenotypic analysis of BM derived MDSC (gated on Gr-1⁺ and CD11b⁺ cells) stained with CD40, CD1d and TLR9 antibodies (open histograms). Grey histograms indicate no antibody staining

(B) MDSC suppress CFSE labelled OT-I proliferation induced by matured DC. DC from BM culture were matured with LPS (10 μg/ml) or α-GalCer in the presence of iNKT cells and then pulsed with SIINFEKL peptide for 2 h. OT-I splenocytes were cultured with matured DCs (5×10⁴) and in the presence or absence of MDSC (3×10⁴). CFSE labelled OT-I splenocytes alone are shown in green histograms.

(C) Staining with CD1d/α-GalCer tetramers and anti CD3 antibody of iNKT cells in CD11c and Gr-1 depleted BM cultures.

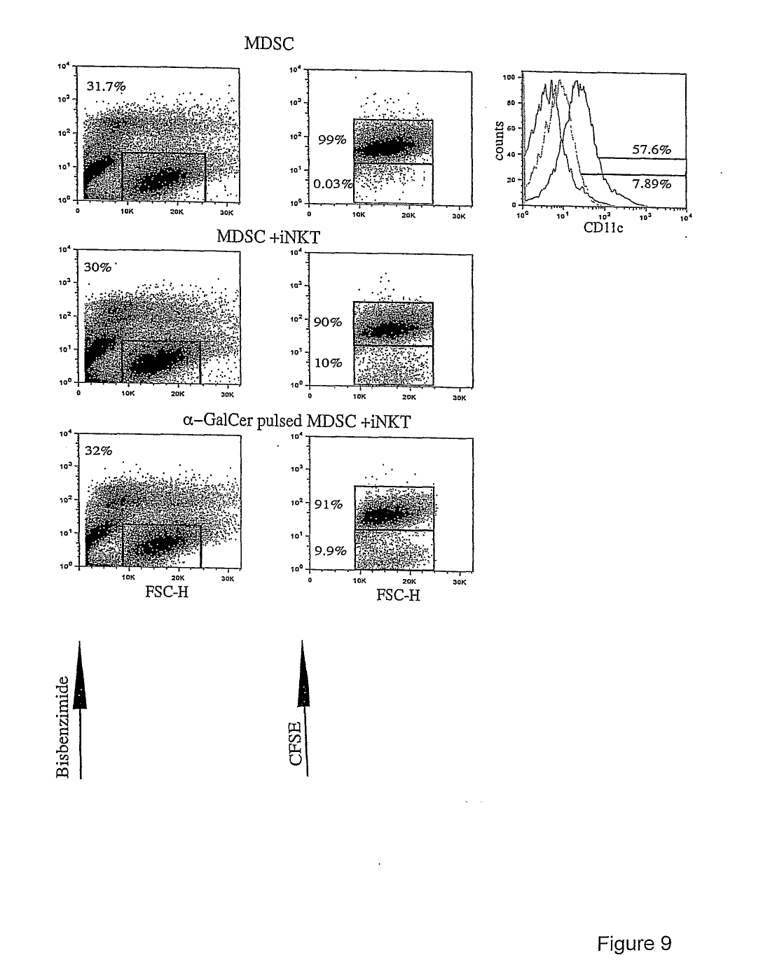

FIG. 9. Lack of iNKT cell dependent lysis of α-GalCer pulsed MDSC.

CFSE labelled BM derived MDSC were either pulsed or not pulsed with α-GalCer and incubated with CD11c and Gr-1 depleted BM cultures, which contain iNKT cells. The percentage of Bisbenzimide negative CFSE labelled MDSC is very similar in the α-GalCer pulsed and unpulsed cultures, indicating that MDSC pulsed with 100 ng of α-GalCer were not sensitive to killing by iNKT cells. As a positive control for iNKT cell activity, the right panel shows CD11c expression on CFSE positive and Bisbenzimide negative MDSC, 48 h after incubation with iNKT cells. Blue histogram shows CD11c expression on α-GalCer pulsed CFSE labelled MDSC in the presence of iNKT cells, while green histogram shows CD11c expression on unpulsed CFSE labelled MDSC in the presence of iNKT cells. Red histogram shows CD11c expression on unpulsed CFSE labelled MDSC in absence of iNKT.

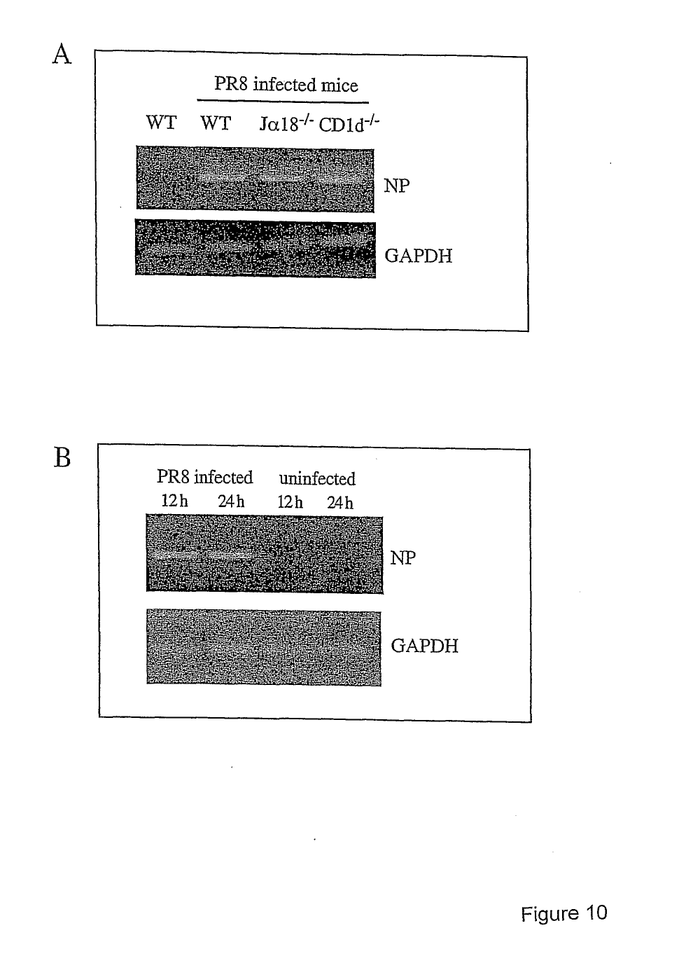

FIG. 10. MDSC can be infected by PR8 virus.

(A) MDSC from lungs of WT, Jα18^{-/-} and CD1d^{-/-} mice infected i.n. with PR8 (3×10⁴ pfu/10⁶ cells) were purified with anti-CD11b-coated magnetic beads. The cells were analysed using semi-quantitative PCR.

(B) BM derived MDSC from WT mice were infected with PR8 (2.5×10⁴ pfu/10⁶ cells) for 1 h. Cells were analysed for expression of PR8 nucleoprotein (NP) 12 hrs and 24 hrs later using semi-quantitative PCR.

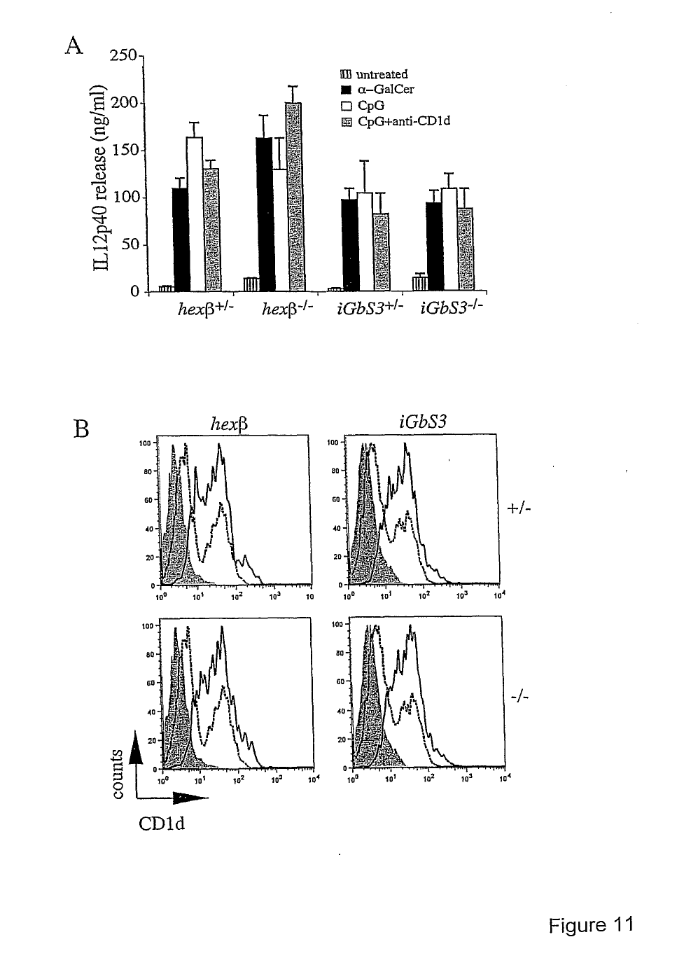

FIG. 11. Phenotypic changes of CpG treated MDSC.

(A) CpG can induce IL-12p40 secretion from hexβ^{-/-} and iGb3S^{-/-} MDSC in the presence or absence of anti CD1d antibody. Supernatants were harvested at 48 h.

(B) MDSC derived from BM of hexβ^{-/-}, iGb3S^{-/-} and iGb3S^{-/-} mice were treated with CpG for 24 h. Treated and untreated MDSC were stained with anti CD1d-PE antibody.

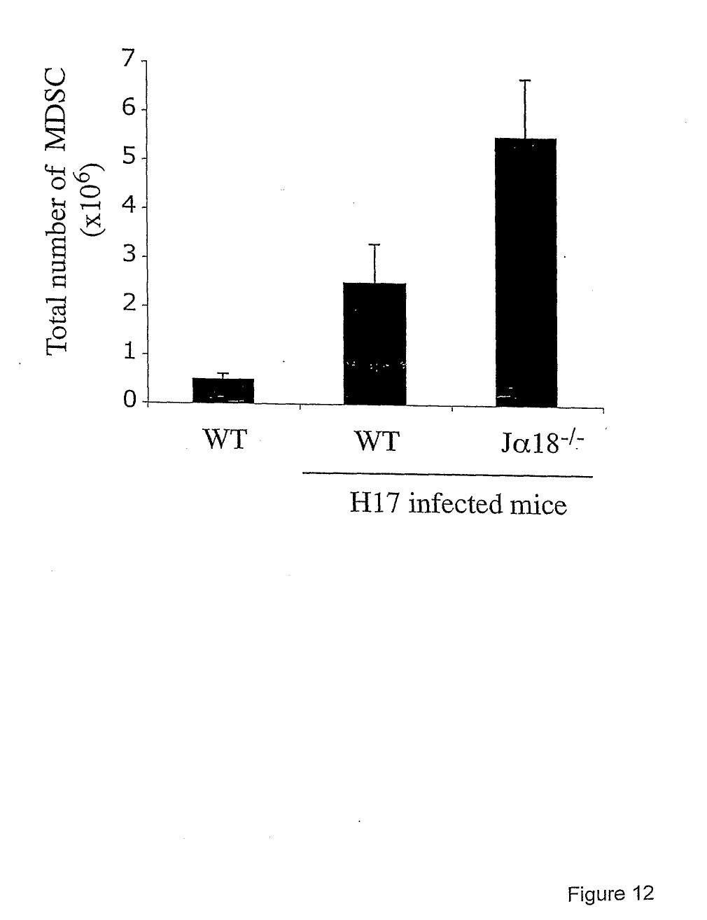

FIG. 12. H17 IAV (H3N2) infection induces a greater expansion of MDSC in Jα18^{-/-} mouse than in WT mice. Expansion of lung infiltrating CD11b⁺ Gr-1⁺ cells in Jα18^{-/-} and in WT mice injected i.n. with H17 IAV (20 HAU), which peaked at approximately day 9 after the infection. Data represents the average of n=5 mice/group±S.D.

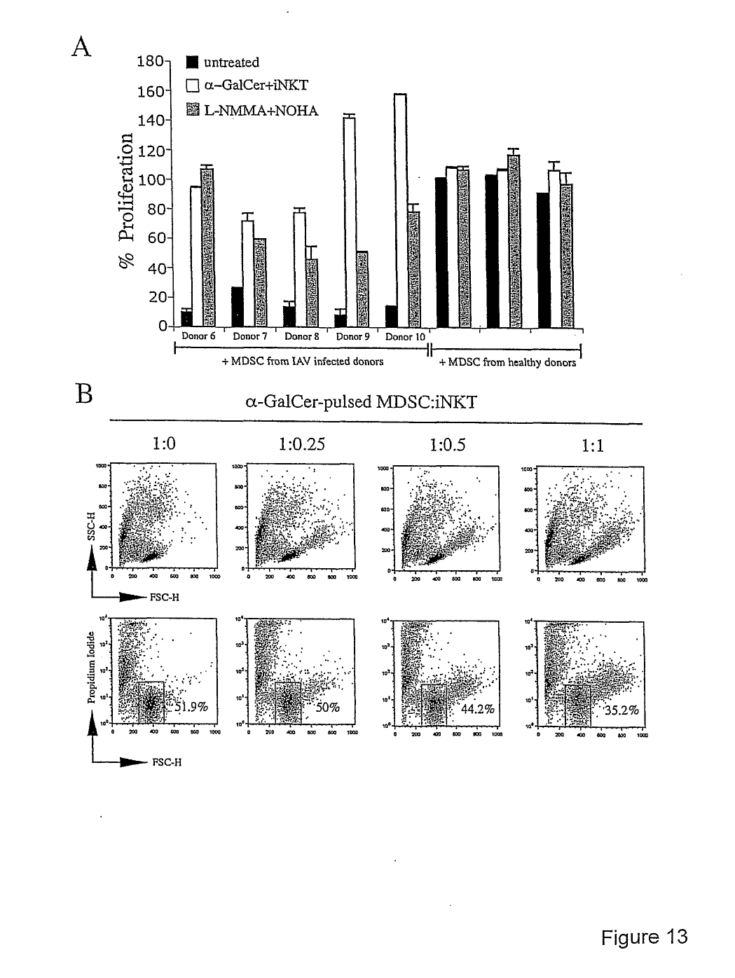

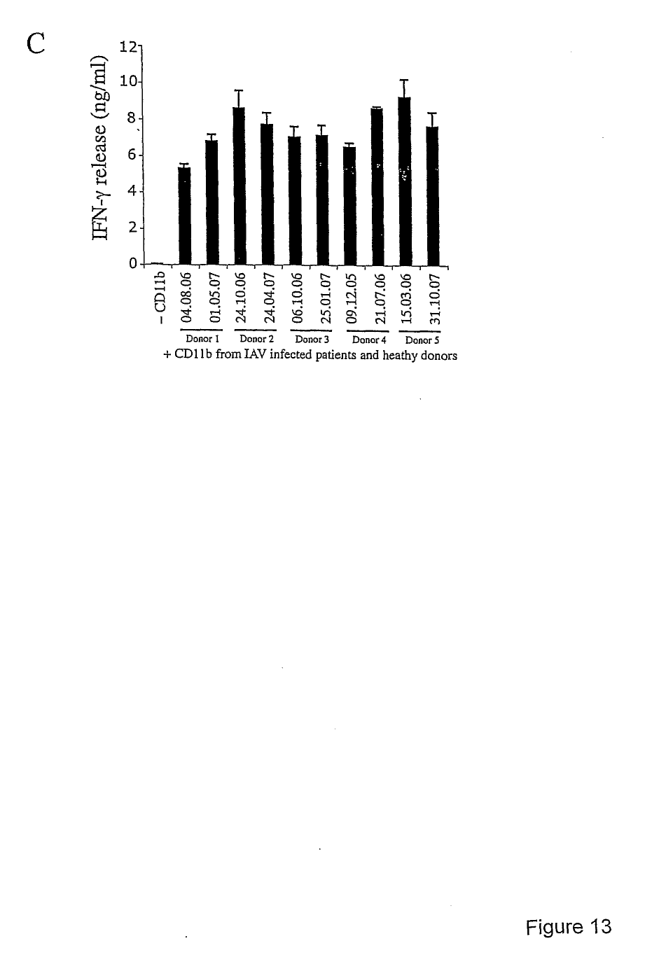

FIG. 13. Inhibition of T cell proliferation by human MDSC can be rescued by iNKT cells.

(A) CD11b⁺ cells, purified from PBL either from individuals shortly after an acute respiratory illness (associated with the presence of high IAV specific Ab titer shown in Table 1) or from healthy donors were added to PBL and incubated with allogeneic irradiated DC. Purified CD11b⁺ cells were either untreated (▪), or treated with either α-GalCer (100 ng/ml) in presence of iNKT at a MDSC:iNKT cell ratio of 1:0.25 (□) or L-NMMA and NOHA ( [symbol] ). The data are expressed as described in Methods. Addition of either iNKT cells or L-NMMA and NOHA to the alloreactive T cells in the absence of CD11b⁺ cells did not affect T cell proliferation (data not shown). The ratio of irradiated DC to purified irradiated CD11b⁺ cells was 1:1.

(B) Conditions to prevent lysis of α-GalCer pulsed human MDSC by iNKT cells. Human MDSC (0.5×10⁶) were treated with α-GalCer (100 ng/ml) and co-cultured with increasing numbers of iNKT cells for 24 h. MDSC were stained with Propidium Iodide to calculate the percentage of iNKT cell dependent MDSC killing.

(C) iNKT cells are activated by α-GalCer pulsed CD11b⁺ cells from IAV infected patients. IFN-γ release from iNKT cells incubated for 24 h with α-GalCer pulsed CD11b⁺ cells, derived from the IAV infected patients and healthy controls described in FIG. 7. Data represent the average of 3 replicates ±S.D.

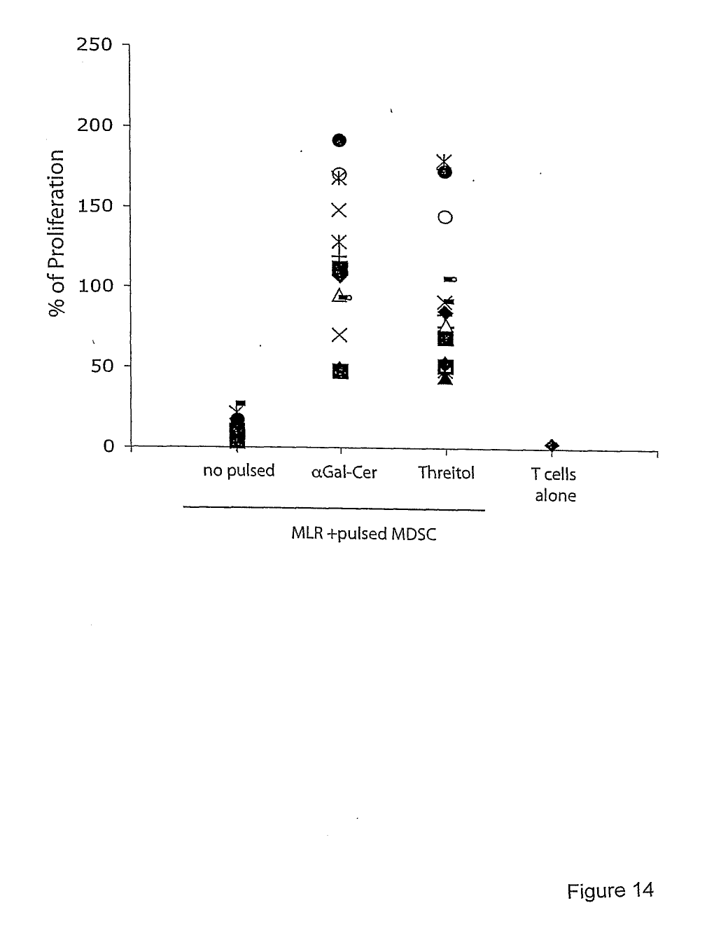

FIG. 14. Expansion of human MDSC during melanoma.

Percentage of T cell proliferation driven by allogeneic irradiated DC in the presence of third party irradiated CD11b+ cells, as compared to alloreactive T cell proliferation in the absence of CD11b+ cells (100% proliferation). Each symbol corresponds to one patient and the results are colour coded (i.e. MDSC from the same patient are represented with the same symbol in the three columns).

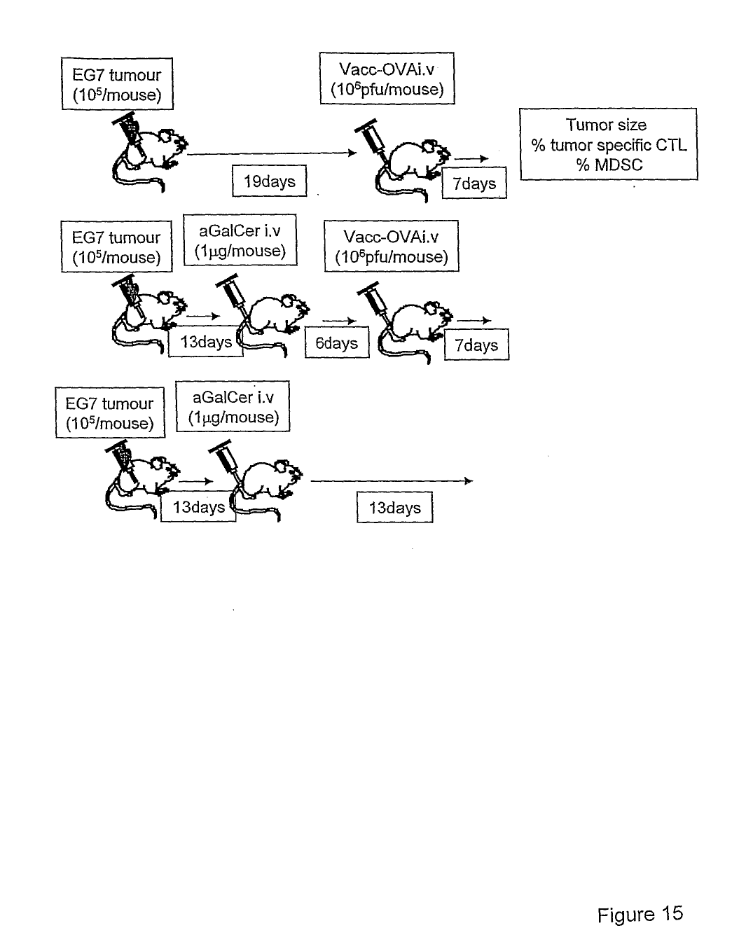

FIG. 15. Experimental protocol for the pre-conditioning of mice with high levels of MDSC.

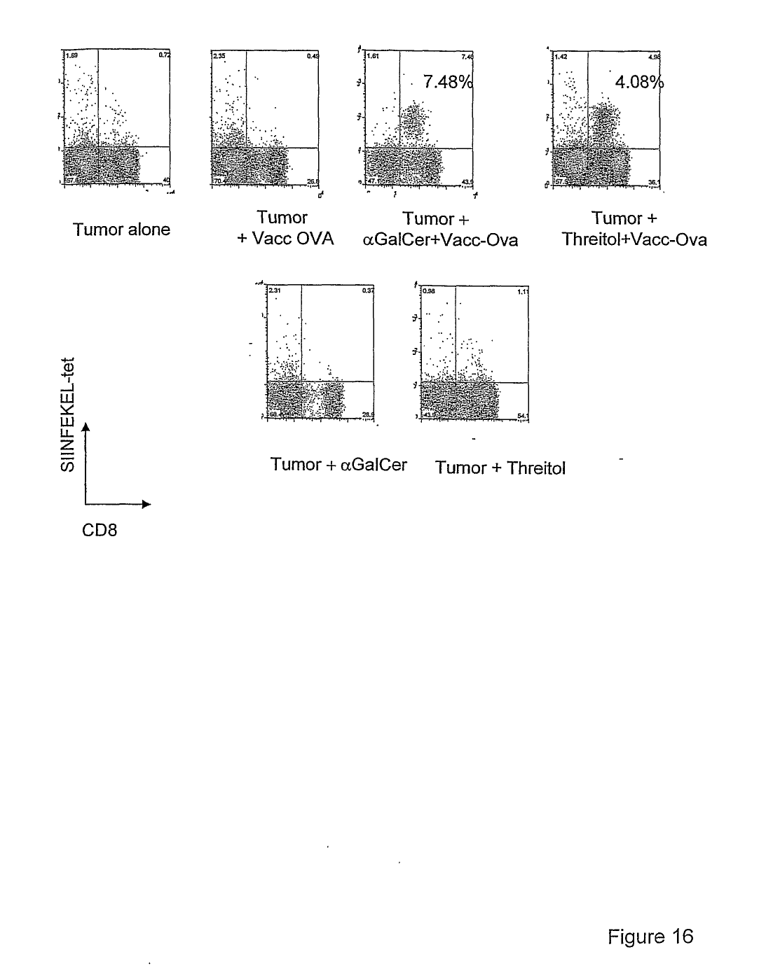

FIG. 16. The frequency of tumor specific CD8+ T cell responses in tumor bearing mice.

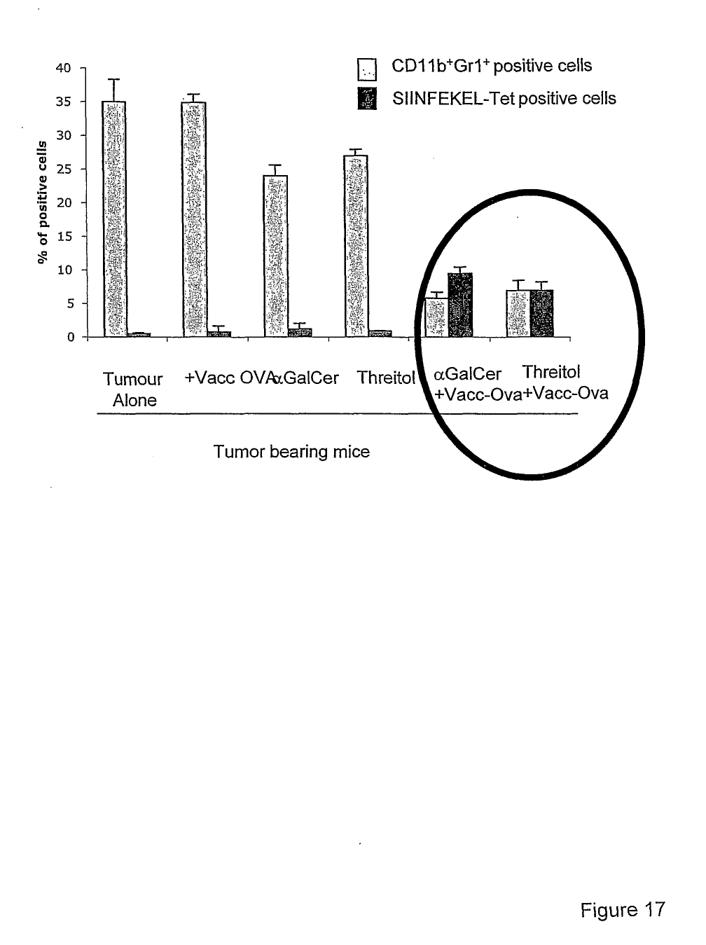

FIG. 17. The frequency of Myeloid Derived Suppressor Cells (CD11b+Gr1+ positive cells) in tumor bearing mice.

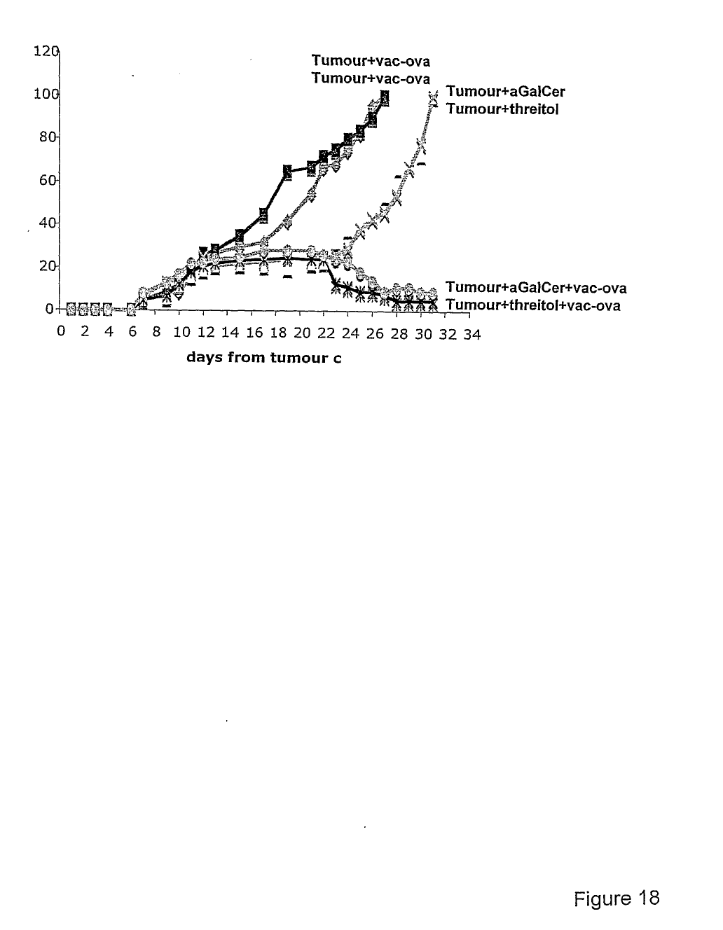

FIG. 18. Tumor regression in tumor bearing mice.

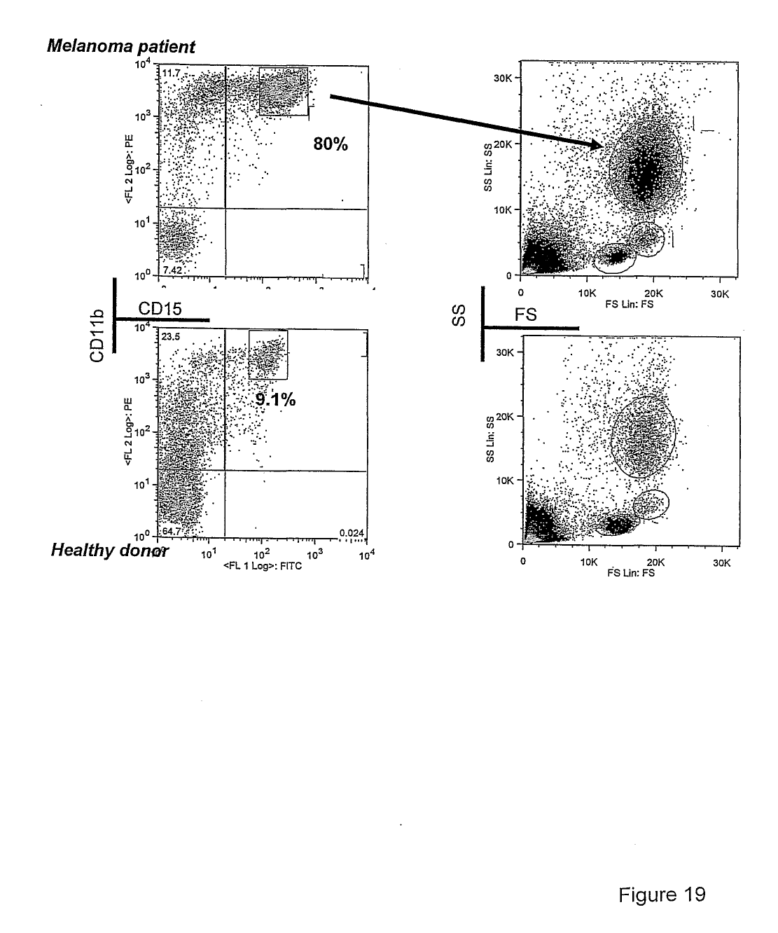

FIG. 19. An enhanced frequency of CD11b+ CD15+ cells in melanoma patients' peripheral blood.

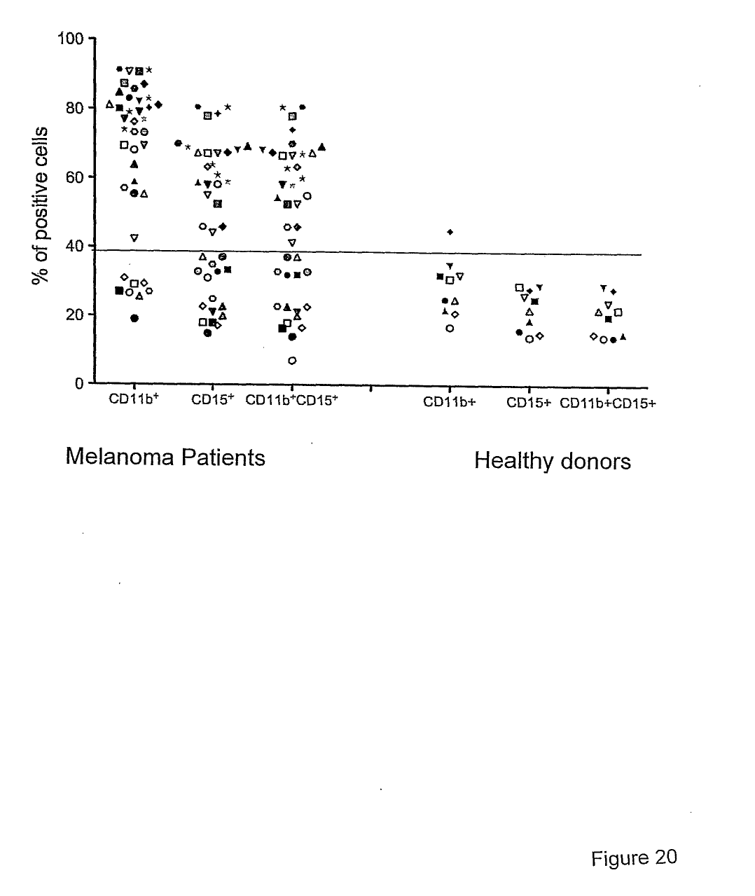

FIG. 20. An enhanced frequency of CD11b+ CD15+ cells in stage III melanoma patients' peripheral blood as compared to healthy patients.

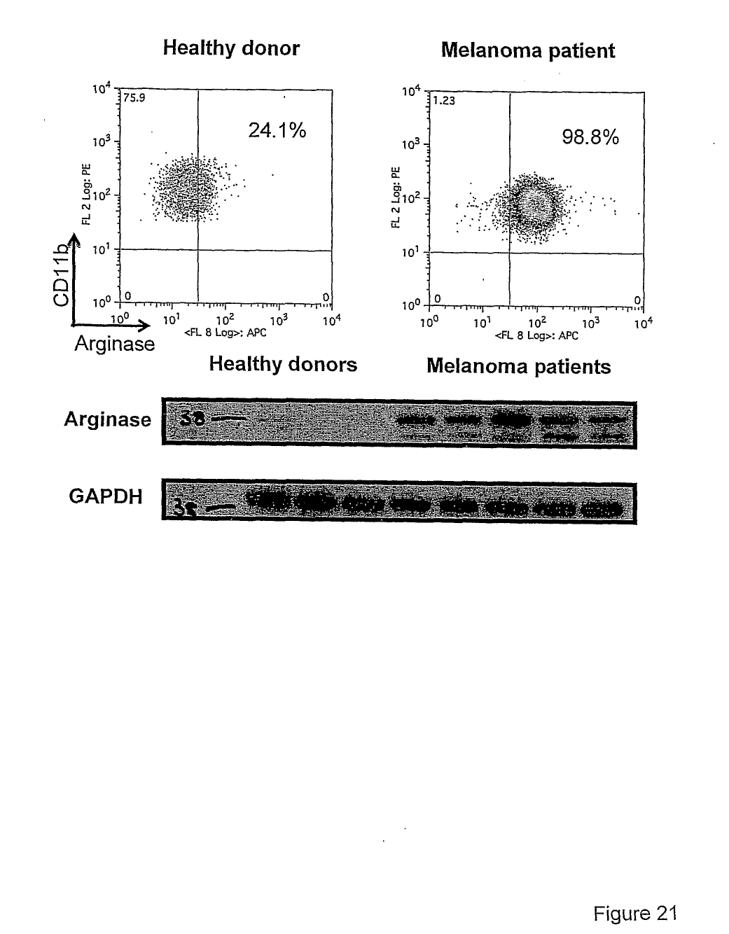

FIG. 21. Arginase I expression by CD11b+ CD15+ cells from melanoma patients.

Upper panel shows expression as measured by intracellular staining. Lower panel shows expression as measured by Western blot analysis.

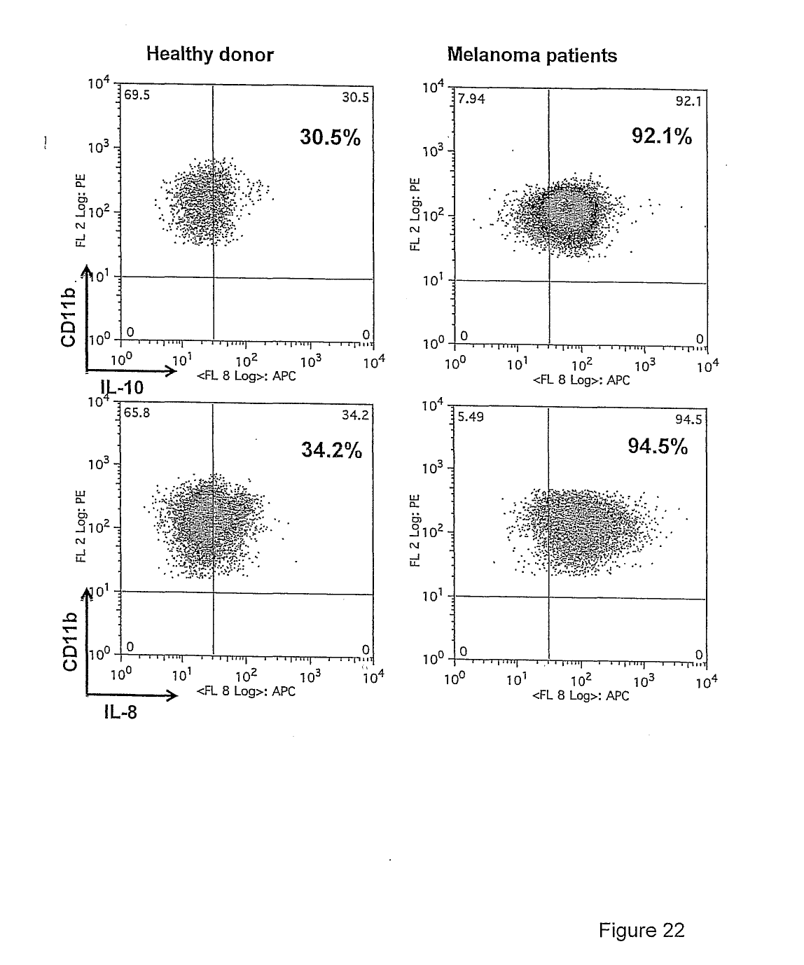

FIG. 22. Secretion of IL-10 (upper panels) and IL-8 (lower panels) from CD11b+ cells from healthy donors (left panels) and melanoma patients (right panels).

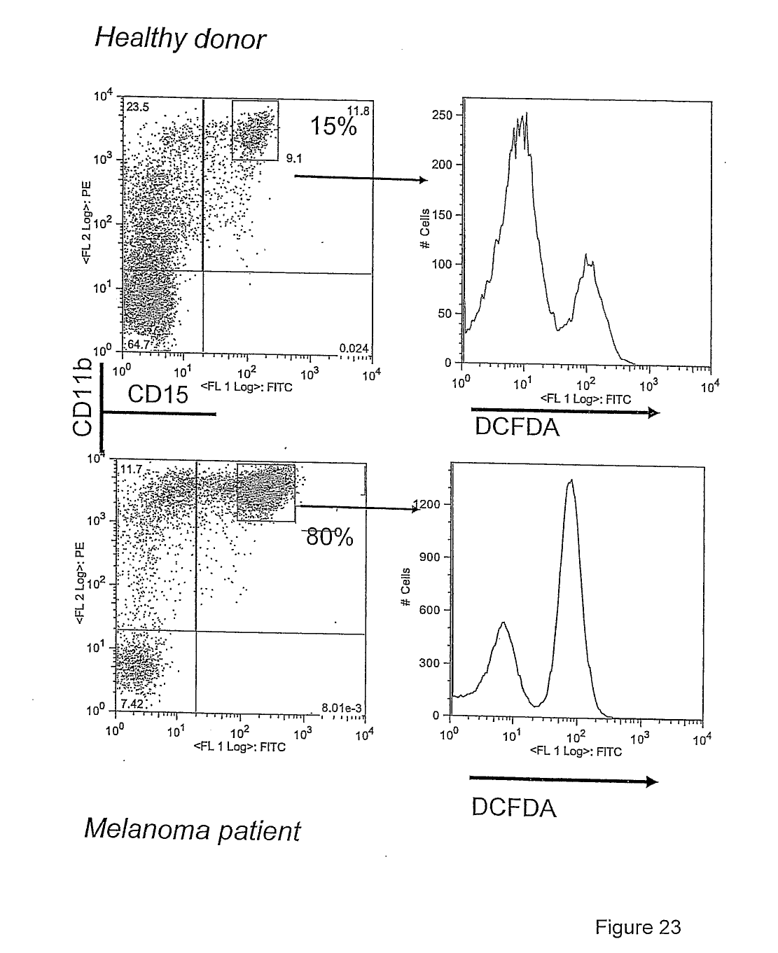

FIG. 23. Secretion of Reactive Oxygen Species (ROS) from healthy donors (upper panels) and melanoma patients (lower panels).

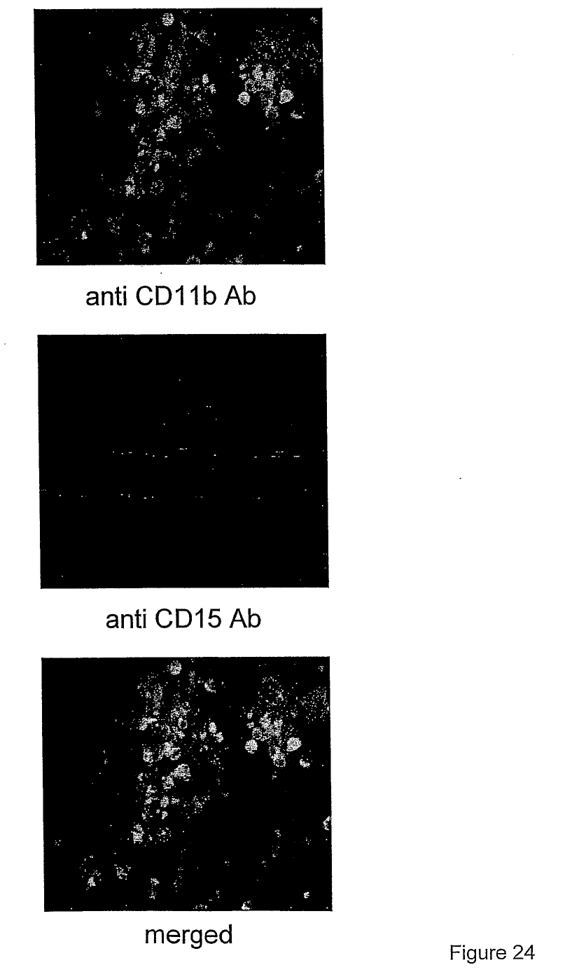

FIG. 24. The presence of CD11bCD15 positive cells inside tumors.

The staining of a primary melanoma section with CD11b (top panel) and CD15 (middle panel) specific antibodies. The lower panel is a merged image of the top and middle panels.

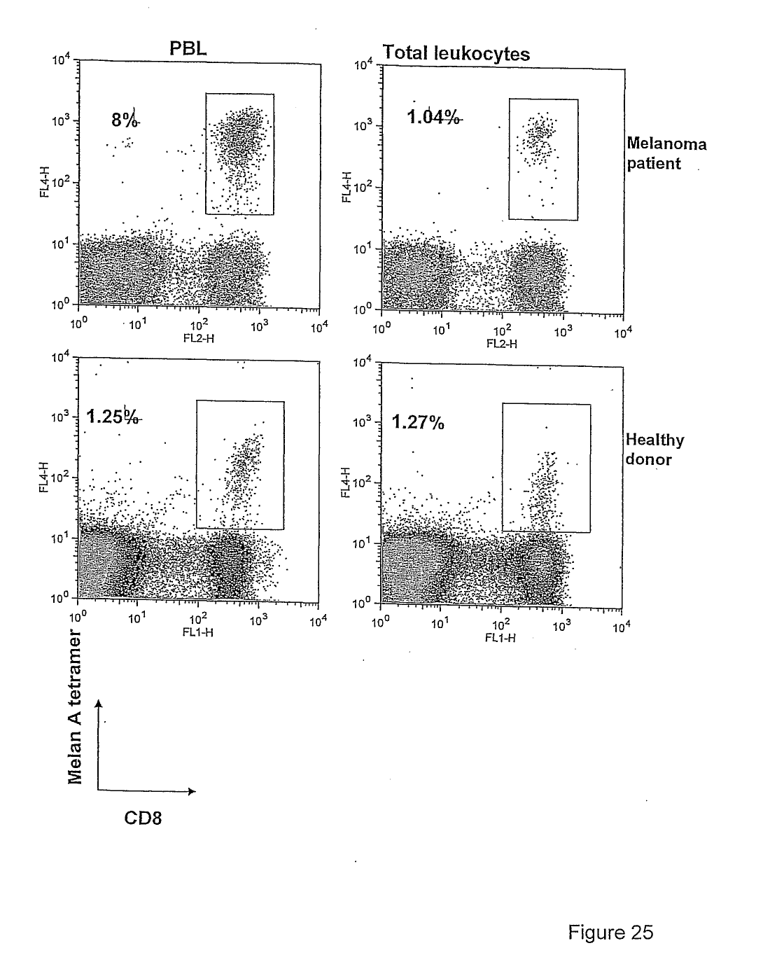

FIG. 25. Reduced expansion of Melan-A26-35 specific T cells in total leukocytes.

FIG. 26. Expansion of Melan-A 26-35 specific CD8+ T cells from peripheral blood of melanoma patients.

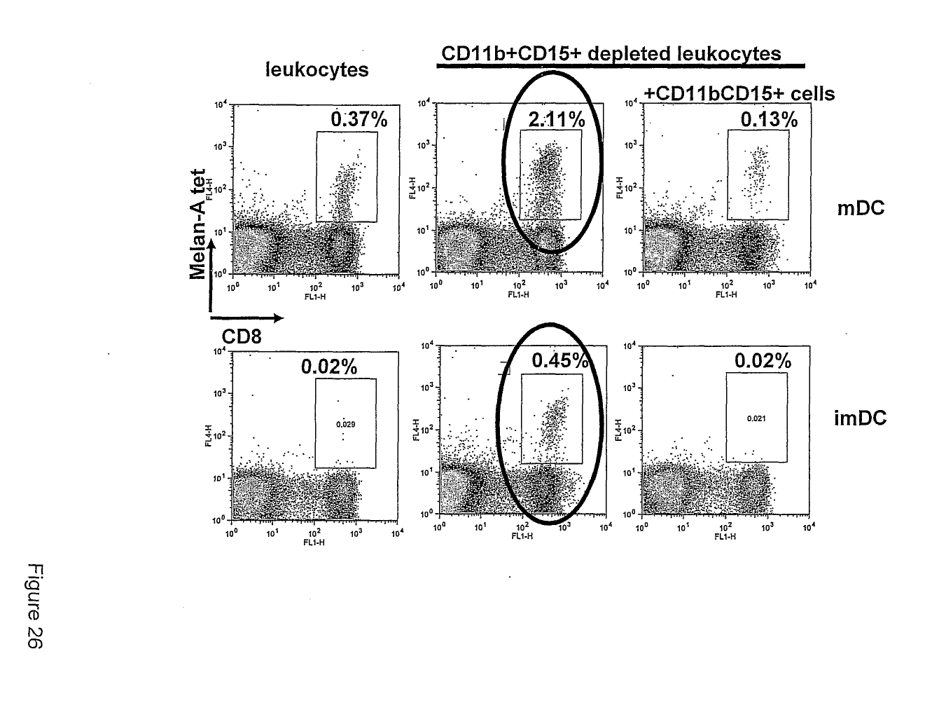

FIG. 27. CD11b/CD15+ cells from healthy donors fail to inhibit Melan-A26-35 T cell priming.

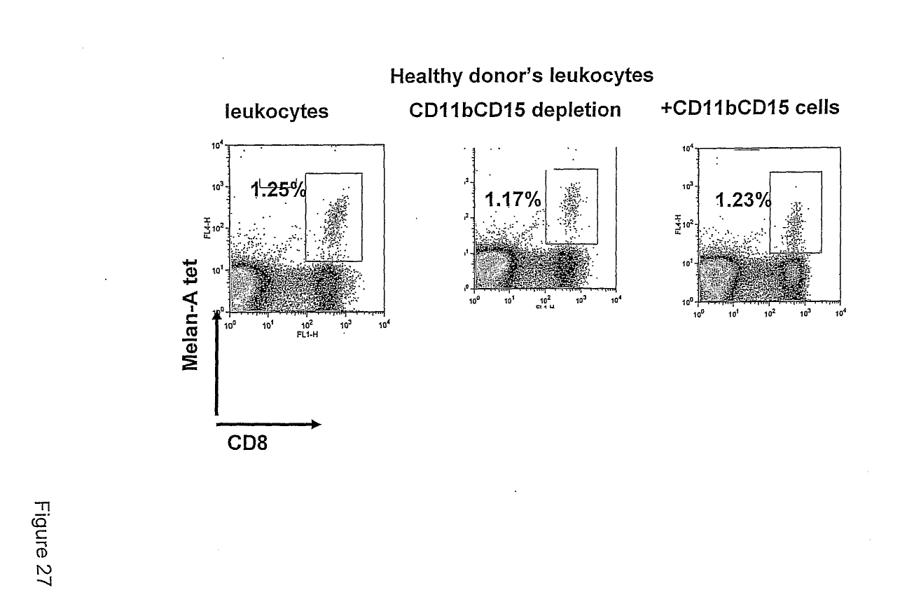

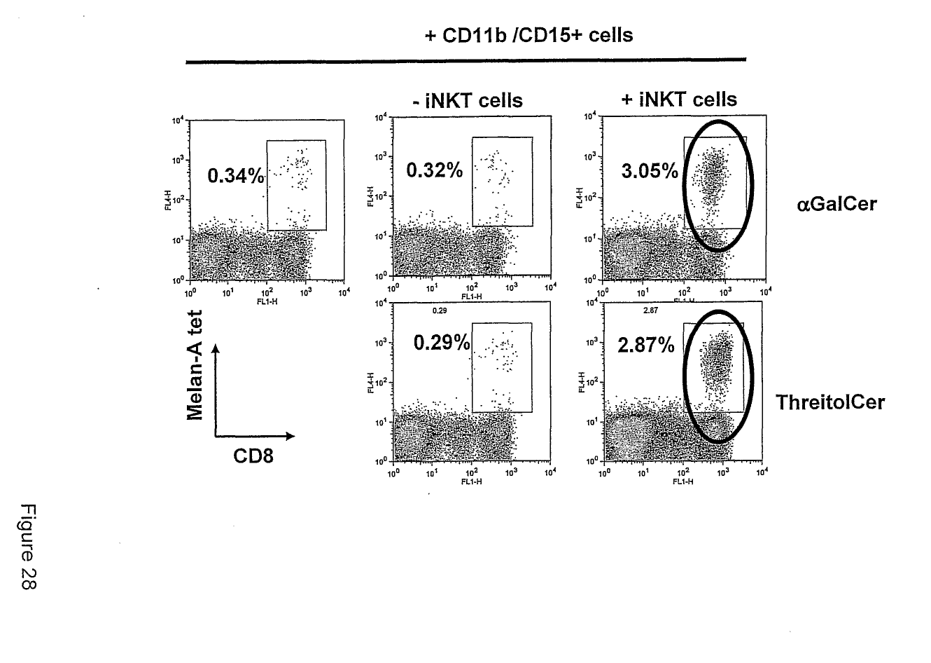

FIG. 28. Harnessing iNKT cells abolishes MDSC suppressive activity and restores Melan-A26-35 specific CD8+ T cell response.

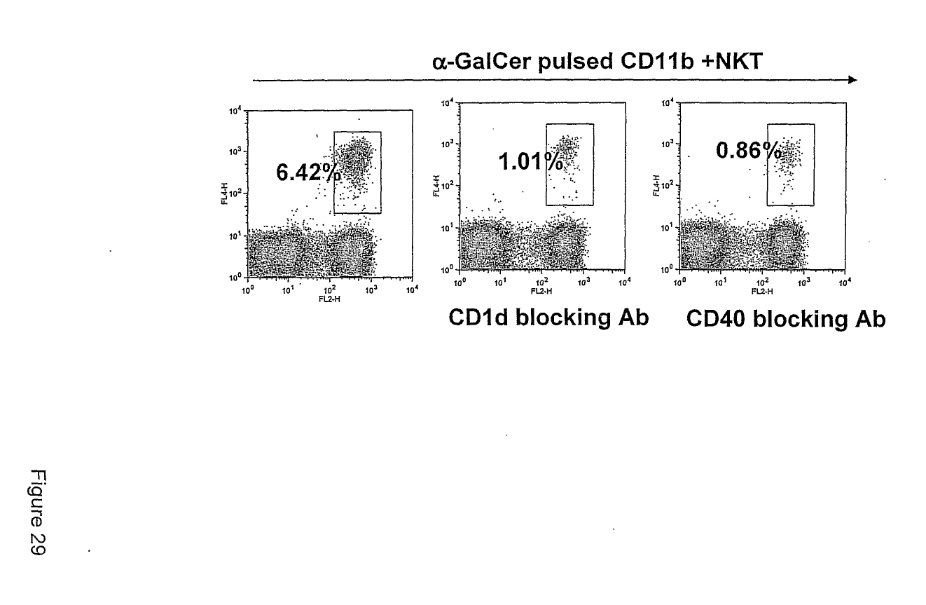

FIG. 29. The ability of iNKT cells to relieve the suppressive activity of CD11bCD15 positive cells is CD1d- and CD40-dependent.

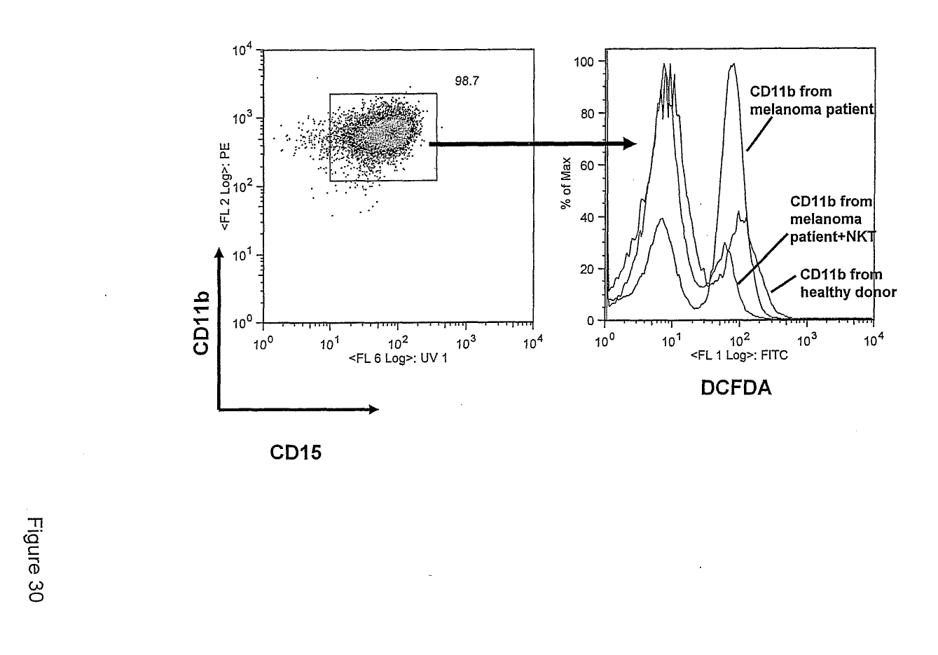

FIG. 30. ROS levels in purified MDSC from melanoma patients are reduced by co-incubation with iNKT cells.

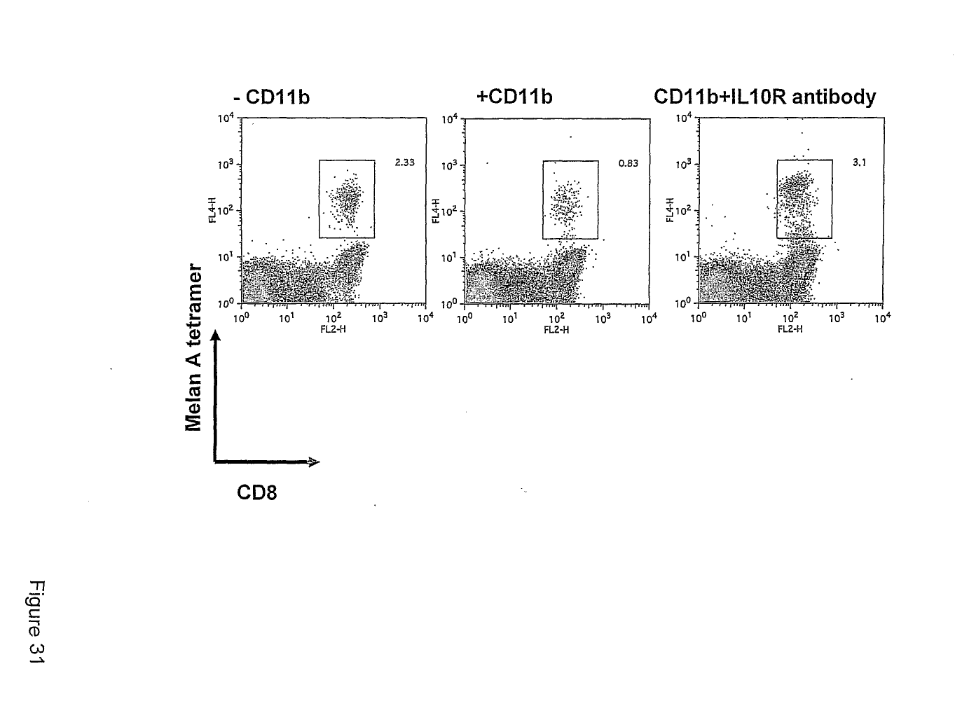

FIG. 31. The suppressive effect of Cd11bCd15+ cells is mediated by the release of IL-10.

### EXAMPLE 1

### iNKT Cell Dependent Resistance to Lethal Intranasal Injection of the IAV A/Puerto Rico/8/34

To assess whether iNKT cells play a role in controlling IAV infection, iNKT deficient (Jα18^{-/-}) and CD1d^{-/-} mice were infected with the IAV A/Puerto Rico/8/34 (PR8). We observed that injection of high doses of PR8 (3×10⁴ pfu) into JA18^{-/-} and CD1d^{-/-} mice resulted in increased mortality, as compared to the mortality rate of PR8 infected wild type (WT) mice (FIG. 1A). Consistent with these results, PR8 infected JA18^{-/-} and CD1d^{-/-} mice had higher IAV titers (FIG. 1B), and reduced percentage and absolute numbers of both PR8 specific CD8+ T lymphocytes (FIG. 1C and data not shown) and antibodies (FIG. 1D), when compared with PR8 infected WT mice. Since iNKT cell frequency was enhanced in the lungs of PR8 infected WT mice (data not shown), we assessed whether adoptive transfer of iNKT cells into PR8 infected JA18^{-/-} mice could enhance survival rate. We observed that the adoptive transfer of iNKT cells into PR8 infected JA18^{-/-} mice rescued the survival of a large proportion of infected mice (FIG. 1A) and was associated with an increase in the frequency and absolute numbers of PR8 specific T cell and antibody responses (FIGS. 1C and 1D, and data not shown) and with a reduction of IAV titer (FIG. 1B). As a control, we showed that the injection of iNKT cells into PR8 infected CD1d^{-/-} mice failed to protect them from PR8 infection and did not have any effect on PR8 virus titer and PR8-specific immune responses (FIG. 1).

These results indicate that iNKT cells play an important role in controlling influenza A PR8 virus infection and that this effect is CD dependent.

### EXAMPLE 2

### Adoptive Transfer of iNKT Cells Significantly Reduces the Suppressive Activity of PR8 Expanded MDSC

It has previously been shown that lungs of IAV infected mice are infiltrated by neutrophils, expressing CD11b and Gr-1 markers. Since it is known that alteration of cytokines during acute and chronic inflammations results in the expansion of ARG-1 and iNOS expressing MDSC (i.e. CD11b⁺ Gr-1⁺ cells) and since cytokines and chemokines are elevated during influenza virus infection, we measured the frequency and activity of CD11b⁺ Gr-1⁺ cells in the lungs of PR8 infected WT, JA18^{-/-} and CD1d^{-/-} mice (FIG. 2A). We observed an expansion of the percentage and absolute numbers of CD11b⁺ Gr-1⁺ cells in PR8 infected mice, which was greater in JA18^{-/-} and CD1d^{-/-} infected mice than in WT infected mice (FIG. 2A and data not shown). Importantly, injection of iNKT cells in JA18^{-/-} mice, but not in CD1d^{-/-} mice, resulted in the reduction of CD11b⁺ Gr-1⁺ cells (FIG. 2A). Phenotypic analysis demonstrated that a large proportion of PR8 induced CD11b⁺ Gr-1⁺ cells in WT, JA18^{-/-} and CD1d^{-/-} mice, expressed CD1d, CD40 and the neutrophil marker 7/4 (data not shown). Injection of lower doses of PR8 (3×10² pfu), while confirming the higher frequency of CD11b⁺ Gr-1⁺ cells in JA18^{-/-} than in WT mice, showed that JA18^{-/-} mice were capable of controlling sublethal PR8 infection and that the numbers of PR8 induced CD11b⁺ Gr-1⁺ cell declined to background levels by day 30 after the infection (FIG. 2B).

To investigate the functional activity of CD11b⁺ Gr-1⁺ cells, we set up a proliferation assay using CFSE labelled OT-I cells as responders. We demonstrated that, while CD11b⁺ Gr-1⁺ cells purified from PR8 infected WT mice did not have a strong suppressor activity, CD11b⁺Gr-1⁺ cells isolated from the lungs of PR8 infected JA18^{-/-} and CD1d^{-/-} mice suppressed in vitro proliferation of CFSE labelled OT-I splenocytes (FIG. 2C). In addition, their adoptive transfer into naïve mice inhibited the ability of recipients to mount antigen specific immune responses (FIG. 2E). To identify the mechanisms controlling PR8 induced CD11b⁺ Gr-1⁺ cell mediated suppressive activity, we established that OT-I proliferation could be rescued by treating CD11b⁺ Gr-1⁺ cells either with inhibitors of ARG1 and NOS activity or by incubating them with anti-CD40 agonist antibody (FIG. 2D). Finally, adoptively transferring iNKT cells into JA18^{-/-} mice significantly reduced both in vitro and in vivo the suppressive activity of PR8 induced CD11b⁺ Gr-1⁺ cells, while adoptive transfer of iNKT cells into CD1d^{-/-} mice had no effect on the suppressive activity of PR8 induced CD11b⁺ Gr-1⁺ cells (FIGS. 2C and E).

These results demonstrate that lungs of JA18^{-/-} and CD1d^{-/-} mice infected with PR8 have a higher numbers of CD11b⁺ Gr-1⁺ cells, which are functionally similar to MDSC, shown to be expanded during acute and chronic inflammatory processes. Our results also identify the suppressive mechanisms by which lung infiltrating CD11b⁺ Gr-1⁺ cells inhibit T cell proliferation and highlight a direct link between the presence of iNKT cells in PR8 infected mice and lack suppression by CD11b⁺ Gr-1⁺ cells.

### EXAMPLE 3

### The Ability of iNKT Cells to Abolish the Suppressive Activity of MDSC is CD40/CD40L Dependent

In order to further study the cross-talk between iNKT cells and CD11b⁺ Gr-1⁺ cells, and understand the mechanisms by which iNKT cells abolish PR8 induced suppressive activity of CD11b⁺ Gr-1⁺ cells, further experiments were carried out using bone marrow (BM) derived CD11b⁺ Gr-1⁺ cells, expressing ARG1 and NOS2. We confirmed that BM derived CD11b⁺ Gr-1⁺ cells express CD1d and CD40 (FIG. 8A) and have ARG1 and NOS2 activity, as defined by their ability to generate urea and peroxynitrates (FIG. 3A, time point 0). We also demonstrated that BM derived CD11b⁺ Gr-1⁺ cells were capable of inhibiting in vitro proliferation of splenocytes from OT-I mice (FIG. 3B, panel a), even in the presence of peptide pulsed matured DC (FIG. 8B). Since we demonstrated that GM-CSF treated BM derived CD11b⁺ Gr-1⁺ cells have a suppressive activity, they will be referred hereafter as to MDSC. Pulsing MDSC with 100 ng of the iNKT cell agonist α-GalCer in the presence of iNKT cells (FIG. 8C) led to the reduction of ARG1 and NOS2 activity and to the enhanced secretion of IL-12 p40 (FIG. 3A). No secretion of IL-23 and IL-12p70 was observed (data not shown). Although previous reports have demonstrated that IL-12p40 reduces IL-12-mediated Th1 responses in vivo by blocking the binding of IL-12 or IL-23 to their receptors, it is unlikely that the enhanced secretion of IL-12p40 by MDSC either treated with TLR-L (see below) or incubated with iNKT cells had an inhibitory effect, as defined by the in vivo and in vitro enhanced immune responses. As a control, we confirmed that MDSC pulsed with 100 ng of α-GalCer were not lysed by iNKT cells (FIG. 9) and that incubation of CFSE labelled α-GalCer pulsed CD11c⁻ MDSC with iNKT cells led to their differentiation into CD11c⁺ cells (Supplementary FIG. 2) and up-regulation of CD86 and MHC class II expression (data not shown),

We further investigated the effect of α-GalCer and BM cultures on MDSC suppressive activity by assessing the effect of MDSC on CFSE labelled OT-I proliferation (FIGS. 3B and C). We showed that pulsing MDSC with α-GalCer in the presence of BM resident iNKT cells from WT mice (FIG. 8C), completely relieved MDSC suppression (FIG. 3B, panel d), as compared to BM derived from JA18^{-/-} (FIG. 3B, panels b and e) and CD1d^{-/-} mice (FIG. 3B, panels c and f). The effect of iNKT cells on MDSC suppressive activity was CD40 and CD40L dependent, as defined by the results of experiments carried out with CD40^{-/-} and CD40L^{-/-} mice (FIG. 3C, panels e and f) and the use of agonist anti CD40 Ab (FIG. 3C, panel i and h).

Collectively, our findings demonstrate that the in vitro activation of iNKT cells by α-GalCer pulsed MDSC leads to phenotypic and functional differentiation of MDSC, events which are dependent on CD40-CD40L interaction.

### EXAMPLE 4

### TLR-L Treatment and IAV Infection of MDSC Relieves MDSC Suppressive Activity

We then assessed whether the effect of TLR-L and IAV infection of MDSC suppressive activity, in the presence or absence of iNKT cells. We showed that incubation of MDSC with TLR3 (poly I:C), TLR7/8 (R848) and TLR9 (CpG 2216) agonists, in the absence of iNKT cells, partially relieved MDSC suppressive activity on CFSE labelled OT-I proliferation (FIG. 4A) and resulted in IL-12 p40 secretion (data not shown). Consistent with previous findings demonstrating TLR-L dependent cross-talk between DC and iNKT cells, we showed that addition of iNKT cells to TLR-L-treated MDSC further reduced MDSC dependent suppressive activity (FIG. 4A) and enhanced IL-12p40 production (data not shown). It is worth noting that LPS failed to relieve MDSC suppressive activity, at a broad range of different concentrations, both in the presence and in the absence of iNKT cells (data not shown). Subsequent experiments demonstrated that CpG and poly I:C treated MDSC were capable of activating iNKT cells and that such activation was CD1d dependent, as defined by the lack of IFNγ and IL-4 secretion in the presence of anti-CD1d blocking antibodies (FIG. 4B).

The capacity of a range of TLR-L to reduce MDSC dependent suppressive activity raised the possibility that the ability of iNKT cells to reduce the suppressive activity of CD11b⁺ Gr-1⁺ cells in PR8 infected mice could be accounted for by a direct cross-talk between PR8 infected MDSC and iNKT cells. Consistent with the observation that lung infiltrating CD11b⁺ Gr-1⁺ cells purified from PR8 infected mice are infected by PR8 virus (FIG. 10A), we showed that BM derived MDSC could be infected by PR8 (FIG. 10B) and that PR8 infected MDSC were capable of activating iNKT cells, as defined by the secretion of IFNγ and IL-4 (FIG. 4C).

These results are consistent with the observation that TLR-L-treated MDSC can foster the interaction with iNKT cells and provide important insights into the mechanisms controlling the cross talk between iNKT cells and MDSC in PR8-infected mice.

### EXAMPLE 5

### MDSC Derived from glycospingolipid Storage Disease Mice Fail to Stimulate iNKT Cells

To further investigate the mechanisms which control the cross-talk between iNKT cells and TLR-L treated MDSC, we used MDSC derived from β-hexosaminidase A/B deficient (hexβ^{-/-}) mice, which are unable to activate iNKT cells due to the accumulation of glycosphingolipids (GSL) in the endo-lysosomal compartment. We showed that the cross-talk between iNKT cells and CpG or poly I:C treated MDSC is compromised in GSL storage disorders (FIGS. 5A and B), although TLR-L incubation of hexβ^{-/-} derived MDSC up-regulates CD1d expression and results in enhanced IL-12 secretion (FIG. 11). The partial proliferation of OT-I splenocytes in the presence of hexβ^{-/-} derived MDSC treated with CpG (FIG. 5B) is due to a direct effect of TLR9 signalling events on MDSC, which is consistent with the results shown in FIG. 4A, indicating that incubation of MDSC with the TLR9 agonist CpG, in the absence of a cross-talk with iNKT cells, partially relieves MDSC suppressive activity on OT-I proliferation. We extended these results by demonstrating that incubation of CpG and poly I:C treated MDSC with the specific inhibitor of GSL biosynthesis, N-butyldeoxygalactonojirimycin (NB-DGJ) reduced the ability of iNKT cells to relieve the suppressive activity of MDSC, as defined by a reduced CFSE labelled OT-I proliferation (FIG. 5C). Consistent with previous findings, these results indicate that TLR-agonists, in addition to a direct maturation effect on MDSC, induce the up-regulation of endogenous iNKT cell ligand(s), which result in the activation of iNKT cells and further relieve of MDSC suppressive activity. Incubation of Hexb^{-/-} derived mice with TLR-L, while failing to facilitate the cross-talk between MDSC and iNKT cells, does not abolish a direct TLR-L maturation effect on MDSC, which results in a partially relieve of MDSC suppressive activity.

Based on investigations in Hexb^{-/-} mice, iGb3 was proposed to be the endogenous ligand essential for the positive selection of mouse iNKT cells in the thymus. Although iGb3 can stimulate iNKT cells in vitro, there is currently no direct evidence supporting the hypothesis that iGb3 plays a role as an endogenous selecting ligand in vivo. Furthermore, iGb3 synthase deficient (iGb3S^{-/-}) mice, with a deletion of the catalytic region of the iGb3S gene, demonstrated that in the absence of detectable isoglobo-series of GSL (iGb3, iGb-4, and iGb-5), the frequency and phenotype of iNKT cells was not altered. We have now extended these results by demonstrating that iGb3 is not necessary for the cross-talk between iNKT cells and CpG-matured MDSC, since CpG-treated MDSC derived from iGb3S^{-/-} were still able to activate in a CD1d dependent manner liver purified iNKT cells from WT mice (FIGS. 5A and B, right panels and FIG. 11A).

These results indicate that the cross-talk between iNKT cells and TLR-L matured MDSC is prevented by conditions in which loading of endogenous ligand(s) onto CD1d molecules is compromised either by the accumulation of glycosphingolipid endo-lysosomal storage or by altering GSL byosynthesis.

### EXAMPLE 6

### Expansion of Human MDSC During IAV Infection

Having characterised the interaction between mouse iNKT cells and MDSC derived from an IAV infection model, we extended these results by assessing whether human iNKT cells have a similar effect on human MDSC. We showed that incubation of healthy donor CD11b⁺ monocytes with low doses of GM-CSF (5 ng/ml) led to their differentiation into cells capable of suppressing T cell proliferation in a mixed lymphocyte reaction (MLR) (FIG. 6A). Consistent with recently published data, a similar inhibition of T cell proliferation was obtained by sorting CD14⁺ cells and incubating them with GM-CSF (data not shown). The suppressive activity of GM-CSF treated CD11b⁺ cells could be pharmacologically relieved by adding inhibitors of ARG1 and NOS2 (FIG. 6A). Interestingly, we showed that the suppressive activity of GM-CSF treated CD11b⁺ cells was relieved by either PR8 infection (FIG. 6B) or by incubation with TLR-L in the presence of added human iNKT cells (FIG. 6C). These results are consistent with our previous findings obtained with mouse MDSC and demonstrate that incubation of human GM-CSF treated CD11b⁺ cells with either IAV or TLR-L enhances their ability to cross-talk with human iNKT cells, thus restoring optimal MLR proliferation.

It has previously been shown that MDSC are expanded in tumor patients. However, it remains unclear whether immunosuppressive monocytes are expanded during viral infections in humans and whether harnessing iNKT cells can abolish their suppressive activity. To address these questions, we collected CD11b⁺ cells from peripheral blood lymphocytes of individuals at two different time points: while they were clinically well (1^{st} time point) and within 30-60 days after respiratory illness onset (2^{nd} time point) (FIG. 7 and Table 1). To prove that the respiratory illness was caused by IAV infection, ex-vivo analysis of the suppressive activity of CD11b⁺ cells was correlated with the Haemagglutination inhibition (HI) antibody titer specific for H3N2 and H1N1 IAV strains, which circulated during the time period over which the human material was collected (Table 1).

The results of these experiments showed that ex-vivo purified peripheral blood CD11b⁺ cells collected at the time of IAV seroconversion, suppressed MLR proliferation (FIG. 7 panel A, B and C), as compared to MLR proliferation obtained with CD11b⁺ cells purified from the same individuals before the respiratory symptoms and in the absence of detectable IAV specific Ab response (Table 1). As a further control, we showed that CD11b⁺ cells collected at different time points from individuals with undetectable IAV specific Ab and without acute respiratory illness symptoms failed to inhibit MLR proliferation (FIG. 7 panel D & E and Table 1). The suppressive activity of CD11b⁺ cells collected from IAV infected individuals could be reduced in vitro by incubating CD11b⁺ cells with either ARG1 and NOS2 inhibitors or with α-GalCer in the presence of iNKT cells (FIG. 7), at a MDSC:iNKT cell ratio of 1:0.25, that did not cause iNKT cell dependent MDSC lysis (FIG. 13B), and yet capable of activating iNKT cells (FIG. 13C). Finally, we collected blood samples from 5 additional individuals within a short time (between 15-30 days) after an acute respiratory illness. Although for these donors we do not have access to blood samples collected before the onset of the acute respiratory illness, we demonstrated the presence of high H3N2 (donor 6 and 10) and H1N1 (donor 7, 8, and 9) IAV specific Ab titers (Table 1) and demonstrated the suppressive activity of CD11b⁺ cells, which was relieved by ARG1 and NOS2 inhibitors or by activating iNKT cells (FIG. 13A).

In conclusion, these results demonstrate the expansion of monocytes with a suppressive phenotype during acute IAV infections, which can be significantly reduced by either inhibiting the activity of ARG1 and NOS2 or by harnessing iNKT cells.

TABLE 1
IAV specific antibody titer from collected blood samples.

| | Date of | Anti H3N2 antibody titer | | Anti H1N1 antibody titer | |
|---|---|---|---|---|---|
| | blood | A/Wisconsin | A/Wellington | A/Solomon | A/New |
| Donors | collection | 67/2005 | 1/2004 | Island/03/2006 | Caledonia/20/92 |
| Donor 1 | 04 Aug. 2006 | <10 | <10 | <10 | <10 |
| | 01 May 2007 | <10 | 64 | <10 | <10 |
| Donor 2 | 24 Oct. 2006 | <10 | <10 | <10 | <10 |
| | 24 Apr. 2007 | 64 | 128 | <10 | <10 |
| Donor 3 | 06 Oct. 2006 | <10 | 16 | <10 | <10 |
| | 25 Jan. 2007 | 32 | 64 | <10 | <10 |
| Donor 4 | 09 Dec. 2005 | <10 | <10 | <10 | <10 |
| | 21 July 2006 | <10 | <10 | <10 | <10 |
| Donor 5 | 15 Mar. 2006 | <10 | <10 | <10 | <10 |
| | 13 Oct. 2007 | <10 | <10 | <10 | <10 |
| Donor 6 | 10 Apr. 2006 | 64 | 128 | <10 | <10 |
| Donor 7 | 11 Jan. 2007 | <10 | <10 | 256 | 128 |
| Donor 8 | 06 Nov. 2007 | <10 | <10 | 128 | 128 |
| Donor 9 | 05 Jan. 2006 | <10 | <10 | 64 | 256 |
| Donor 10 | 10 Aug. 2006 | <10 | 16 | <10 | <10 |

Haemagglutinin inhibition assay using indicated strains of IAV. Blood samples from donors 1, 2 and 3 were collected either before (1^{st} time point) or within 30-60 days (2^{nd} time point) after an acute respiratory illness. Blood samples from donors 4 and 5, were collected at the indicated time points in the absence of any acute respiratory illness.

Blood samples from donors 6, 7, 8, 9 and 10 were collected shortly after (between 15-30 days) an acute respiratory illness. Values shown as <10 indicate undetectable antibody titers.

### EXAMPLE 7

### Expansion of Human MDSC During Melanoma

MDSC purified from Melanoma patients were sorted from PBMC of patients using CD11b beads. α-GalCer or Threitolceramide were added to human MDSC for 24 hours in the presence of iNKT cells. The cells were washed twice and irradiated (5000 rad). MDSC (5×10⁴) were added to a mix of peripheral blood lymphocytes (PBL) (2×10⁵) and allogeneic irradiated (5000 rad) DC (5×10⁴) in 200 ml of RPMI 5% human AB serum in 96-well flat-bottom plates. Cells were incubated at 37° C., 5% CO₂ for 5 days and then 1 mCi/well 3H-thymidine (Perkin Elmer life Sciences, Boston, Mass.) was added for 15-18 h. 3H-thymidine incorporation was measured using a Wallac Microbeta Jet 1450 reader (Perkin Elmer). The data are expressed as the percentage of T cell proliferation driven by allogeneic irradiated DC in the presence of third party irradiated CD11b+ cells, as compared to alloreactive T cell proliferation in the absence of CD11b+ cells (100% proliferation) (FIG. 14). Each symbol corresponds to one patient and the results are colour coded (i.e. MDSC from the same patient are represented with the same symbol in the three columns).

### EXAMPLE 8

### Conditioning of Mice with High Levels of MDSC Facilitates the Subsequent Expansion of Vaccine Driven Antigen Specific T and B Cell Responses

A) C57 BL/6 mice are infected intranasally with sub lethal doses of influenza A virus (IAV). Initial experiments are carried out using the IAV PR8 using doses ranging from 3×10⁴ PFU to 3×10⁵ PFU. A few days after the infection, the frequency and suppressive activity of MDSC is monitored in vitro, using assays as described herein. Mice are then injected either intravenously, intranasally, or subcutaneously with a range of IMM47 doses (from 50 ng to 1 μg per mouse). After 24 hours from the IMM47 injection, the frequency and activity of MDSCs are monitored in vitro, using the protocols described herein. Control experiments are carried out in iNKT cell knock out mice (Jalpha 18 deficient mice) to confirm that the injection of IMM47 results in the reduction of the suppressive activity of MDSCs. 7 days after IMM47 treatment, mice are vaccinated with vaccinia virus encoding influenza nucleoprotein (NP) (1×10⁷ PFU) and the frequency of NP specific T cell responses and IAV titer is assessed. An additional cohort of mice would not be vaccinated with vaccinia virus encoding influenza nucleoprotein (NP) following IMM47 treatment. The experiment is summarised in the table below.

| | | | Assay | IMM47 | Assay | | Assay NP |
|---|---|---|---|---|---|---|---|
| Group | Mice | IAV | MDSC | [iv, in, sc*] | MDSC | vvNP | CTL/Ab |
| 1 | C57 BL/6 | Yes | Yes | Yes | Yes | Yes | Yes |
| 2 | C57 BL/6 | Yes | Yes | No | Yes | Yes | Yes |
| 3 | Jalpha 18^{-/-} | Yes | Yes | Yes | Yes | Yes | Yes |
| 4 | C57 BL/6 | Yes | Yes | Yes | Yes | No | Yes |
| 5 | C57 BL/6 | Yes | Yes | No | Yes | No | Yes |

subgroups of mice will be treated with IMM47 via different routes

A higher frequency of NP specific T cell responses and lower IAV titer is observed in the IAV infected C57 BL/6 mice treated (conditioned) with IMM47 as compared to control IAV infected C57 BL/6 mice not treated with IMM47 (unconditioned) and the control IAV infected, IMM47 treated Jalpha 18−/− mice. This supports the concept that conditioning of mice with high levels of MDSC facilitates the subsequent expansion of vaccine driven antigen specific T and B cell responses.

B) C57 BL/6 mice were injected subcutaneously with 1×10⁵ EG7 cells (EL4 tumor cell line transfected with ovalbumin cDNA). After 13 days, when tumours are palpable, the frequency and activity of MDSCs was assessed in vitro. Mice were then injected either intravenously with 1 μg α-galactosylceramide or threitolceramide and the frequency of MDSCs is monitored. 6 days after α-galactosylceramide or threitolceramide treatment, mice were infected with vaccinia virus encoding full length ovalbumin (10⁶ pfu/mouse) and both the frequency of OVA specific T cells and tumour burden was monitored. An additional cohort of mice was not vaccinated with vaccinia virus encoding full length ovalbumin following α-galactosylceramide or threitolceramide treatment. The control mice (tumor alone, tumor followed by vaccinia ovalbumin or tumor followed by either α-galactosylceramide or threitolceramide) were analysed for comparison. The experiment is summarised in FIG. 15 and below:
1) tumor alone;
2) tumor vaccinia encoding full length ovalbumin;
3) tumor vaccinia encoding full length ovalbumin pre-conditioned with alphaGalCer;
4) tumor vaccinia encoding full length ovalbumin pre-conditioned with threitolceramide;
5) tumor alone followed by alpha-GalCer; and
6) tumor alone followed by threitolceramide.

Three parameters were monitored: a) frequency of ovalbumin specific responses, as defined by staining with H-2K^{b} tetramers loaded with the ovalbumin peptide SIINFEKEL (FIG. 16); b) frequency of CD11b/Gr1 positive cells (MDSC) in the spleen and blood (FIG. 17); and c) tumor size (FIG. 18).

FIG. 16 shows the frequency of tumor specific CD8+ T cell responses in tumor bearing mice and it can be seen that pre-conditioning of the mice with NKT cell agonists enhances these responses. EG7 tumour cells were transfected with full length ovalbumin cDNA and H-2K^{b} restricted ovalbumin specific T cell responses (specific for the SIINFEKEL peptide) were monitored by tetramer staining. A higher frequency of Ova specific T cell responses was observed in the tumor bearing C57 BL/6 mice treated (conditioned) with α-galactosylceramide or threitolceramide as compared to control tumor bearing C57 BL/6 mice not treated with α-galactosylceramide or threitolceramide (unconditioned) and control tumor bearing α-galactosylceramide or threitolceramide treated mice which were not vaccinated with vaccinia virus encoding the full length ovalbumin protein.

FIG. 17 shows the frequency of Myeloid Derived Suppressor Cells (CD11b+Gr1+positive cells) in tumor bearing mice and it can be seen that pre-conditioning with NKT cell agonists reduces the frequency. A lower frequency of MDSCs was observed in the tumor bearing C57 BL/6 mice treated (conditioned) with α-galactosylceramide or threitolceramide as compared to control tumor bearing C57 BL/6 mice not treated with α-galactosylceramide or threitolceramide (unconditioned) (5 mice per group).

FIG. 18 shows tumor regression in tumor bearing mice and it can be seen that pre-conditioning with NKT cell agonists induces tumor regression. Tumour regression was observed in the tumor bearing C57 BL/6 mice treated (conditioned) with α-galactosylceramide (*) or threitolceramide (•) as compared to control tumor bearing C57 BL/6 mice not treated with α-galactosylceramide or threitolceramide (unconditioned, ♦, ▪) (5 mice per group).

This supports the concept that pre-conditioning tumor bearing mice with iNKT cell agonists reduces the frequency of MDSC and results in higher vaccine driven T cell responses. Since we and others have demonstrated that MDSC are expanded not only during cancer growth but also during bacterial and virus infections, it is thought that injection of iNKT cell agonists during bacterial and viral infections will reduce the frequency of MDSC, hence facilitating expansion of vaccine driven virus or bacterial specific immune responses.

### EXAMPLE 9

### ROS Producing CD11b+ CD15+ Cells are Expanded in Melanoma Patients' PBMC and are Capable of Inhibiting Melan-A Specific T Cell Responses

In order to assess whether the results described above could be extended to cancer patients, experiments were carried out using blood samples from melanoma patients. We observed that a large proportion of melanoma patients have an expansion of CD11b/CD15 positive cells, which have a Forward (FS)/Side (SS) scatter corresponding to granulocytes.

FIG. 19 shows an enhanced frequency of CD11b+ CD15+ cells in melanoma patients' peripheral blood (top panels) as compared to healthy patients (bottom panels). Back-gating of CD11b and CD15 positive cells into Forward (FS) and Side (SS) scatter plots indicates that CD11b and CD15 positive cells fall into the granulocyte gate.

FIG. 20 shows an enhanced frequency of CD11b+ CD15+ cells in stage III melanoma patients' peripheral blood as compared to healthy patients. More than 40 blood samples from melanoma patients were stained with either with anti CD11b and CD15 specific antibody alone or double stained with anti CD11b and CD15 specific antibodies. Each symbol corresponds to a different patient's sample. As a control, similar stainings were carried out with healthy volunteers' blood samples. Of importance, since CD11b-CD15 positive cells have a cell density similar to the density of granulocytes, antibody stainings and FACS guided sorting (in follow up experiments) were carried out using whole blood (after lysing red blood cells), rather than using density gradient purified samples.

The results of these experiments demonstrated that purified CD11bCD15 positive cells from melanoma patients are quantitatively and qualitatively different from CD11bCD15 positive cells purified from healthy donors, as defined by: 1) the expression of arginase (FIG. 21); 2) secretion of IL-10 and IL-8 (FIG. 22), and 3) secretion of Reactive Oxygen Species (ROS (FIG. 23).

FIG. 21 shows the expression of arginase in CD11bCd15 positive cells. The upper panel shows arginase expression as measured by intracellular staining and the lower panel shows arginase expression as measured by western blot analysis. A comparison of arginase expression in patients' samples and healthy donors' samples is shown. The percentage values in the upper panel indicate the percentage of CD11b/CD15 positive cells which are arginase positive. Arginase expression by intracellular staining and western blot was assessed in ex-vivo experiments without treating cells with PMA.

FIG. 22 shows the secretion of IL-10 (upper panels) and IL-8 (lower panels) from CD11b+ cells from healthy donors (left panels) and melanoma patients (right panels). It can be seen that a significant larger proportion of melanoma patients' CD11b+ cells synthesize IL-10 and IL-8, as compared to healthy donors' CD11b positive cells. The percentage values in the upper panel indicate the percentage of CD11b/CD15 positive cells, which are IL-10 (top panel) and IL-8 (bottom panel) positive. IL-10 and IL-8 intracellular staining expression was assessed in ex-vivo experiments without treating cells with PMA.

The role of IL-10 secreted by Cd11bCd15 positive cells in suppressing Melan-A 26-35 specific T cell responses is shown in FIG. 31 and discussed in Example 10 below.

FIG. 23 shows the secretion of Reactive Oxygen Species (ROS) from healthy donors (upper panels) and melanoma patients (lower panels). ROS production was assessed in ex-vivo experiments without treating cells with PMA, by incubating cells with the fluorogenic compound DCFDA (Molecular Probes) (see Corzo et al., J. Immunol., 182:5693, 2009) and gating on CD11b CD15 positive cells. The results of these experiments indicate that CD11b CD15 positive cells purified from melanoma patients produce larger amounts of ROS as compared to CD11b CD15 positive cells purified from healthy donors, as defined by the presence of a larger proportion of DCFDA positive cells in melanoma patients' CD11b CD15 positive cells.

FIG. 24 shows the staining of a primary melanoma section with CD11b (top panel) and CD15 (middle panel) specific antibodies. The lower panel is a merged image of the top and middle panels. It can be seen that CD11bCD15 positive cells can also be found inside tumors.

In order to assess the suppressive activity of CD11bCD15 positive cells, we analysed whether they are capable of inhibiting the expansion of melan-A₂₆₋₃₅ specific T cells from melanoma patients. FIG. 25 shows the percentage of Melan-A₂₆₋₃₅ specific T cells expanded in vitro from either whole blood (total leukocytes) or gradient purified cells (PBL) from either melanoma patients or healthy donors' blood samples. T lymphocytes were stimulated in vitro for 14 days with autologous DC pulsed with the Melan-A₂₆₋₃₅ peptide and then the frequency of Melan-A₂₆₋₃₅ specific T cells was assessed by staining cell cultures with anti CD8 antibodies and with HLA-A2 tetramers loaded with the Melan-A₂₆₋₃₅ peptide. Since total leukocytes from melanoma patients, but not from healthy donors, contain a high frequency of CD11bCD15 positive cells, which were removed after cell gradient purification, this experiment allowed us to compare whether the presence or absence of CD11bCD15 positive cells could affect the in vitro expansion of Melan-A₂₆₋₃₅ specific T cells. The results of these experiments indicated a higher frequency of Melan-A₂₆₋₃₅ specific T cells using PBL from melanoma patients' (8%) as compared to the frequency of Melan-A₂₆₋₃₅ specific T cells expanded from the total leukocyte population (i.e. containing CD11bCD15 positive cells) (1.04%). In contrast, expansion of Melan-A₂₆₋₃₅ specific T cells from healthy donors was similar when using PBL and total leukocytes (1.25% vs 1.27%).

These results indicated that cells contained in the melanoma patients' total leukocyte sample, but not in the healthy donors' total leukocyte samples, were inhibiting Melan-A₂₆₋₃₅ specific T cell expansion. In order to address whether this inhibition was due to CD11bCD15 positive cells, experiments were carried out after selectively depleting total leukocyte samples from CD11bCD15 positive cells (FIG. 26). FIG. 26 shows the expansion of Melan-A₂₆₋₃₅ specific CD8+ T cells from peripheral blood of melanoma patients. In the left hand column, expansion of Melan-A₂₆₋₃₅ specific T cells was assessed using total leukocytes (as explained in FIG. 25). In the middle column, CD11b-CD15+ cells were removed from total leukocytes, and then in the right hand column the CD11b-CD15+ cells were added back. mDC and imDC refer to the use of LPS matured dendritic cells (mDC) or immature dendritic cells (imDC). The results indicate that the frequency of Melan-A₂₆₋₃₅ specific T cells from total leukocyte cultures (0.37% and 0.02% in cultures stimulated with mature and immature DC, respectively) was significantly enhanced after removing from the total leukocytes CD11bCD15 positive cells (2.11% and 0.45% in cultures stimulated with mature and immature DC, respectively). In contrast, adding back CD11b-CD15+ cells reduced the frequency of CD11bCD15 positive cells (0.13% and 0.02% in cultures stimulated with mature and immature DC, respectively). These results demonstrate that the presence of CD11bCd15 positive cells in culture inhibits the expansion of Melan-A₂₆₋₃₅ specific T cells.

As a control, experiments similar to the experiments described in FIG. 26 were carried out using healthy donors' total leukocytes. The results of these experiments are shown in FIG. 27, which shows that CD11b/CD15+ cells from healthy donors fail to inhibit Melan-A₂₆₋₃₅ T cell priming.

In conclusion, the experiments shown in FIGS. 26 and 27 demonstrate that only CD11bCD15+ cells derived from melanoma patients have a suppressive activity, as defined by their ability to inhibit expansion of Melan-A₂₆₋₃₅ specific T cells.

### EXAMPLE 10

### Harnessing iNKT Cells Reduces the Suppressive Activity CD11b+ CD15+ Cells and Restores Melan-A₂₆₋₃₅ Specific T Cell Expansion

In order to assess whether the cross-talk between iNKT cells and CD11bCD15 positive cells could abolish the suppressive activity of CD11bCd15 positive cells, experiments were carried out by co-culturing autologous iNKT cells with autologous CD11bCD15 positive cells, which were pulsed with iNKT cell agonists.

FIG. 28 shows that harnessing iNKT cells abolishes MDSC suppressive activity and restores Melan-A₂₆₋₃₅ specific CD8+ T cell response. CD11bCD15 positive cells were pulsed with either alphaGalCer (100 ng) or Threitolceramide (200 ng) for 2 hours and then incubated with autologous iNKT cells overnight before adding autologous DC pulsed with Melan-A₂₆₋₃₅ peptide and autologous CD11bCD15 cell depleted total leukocytes. The results indicate that the frequency of Melan-A₂₆₋₃₅ specific T cells from total leukocyte cultures (0.34%) (left column) was not significantly enhanced after pulsing CD11bCD15 positive cells with either alphaGalCer (0.32%) or Threitolceramide (0.29%) in the absence of added iNKT cells (middle column). In contrast, addition of iNKT cells to either alphaGalCer (3.05%) or Threitolceramide (2.87%) pulsed CD11bCD15 positive cells significantly enhanced the frequency of Melan-A₂₆₋₃₅ specific T cells (right column).

Finally we showed that the ability of iNKT cells to relieve the suppressive activity of CD11bCD15 positive cells is dependent on CD1d and CD40 expression by CD11bCD15 cells, as defined by experiments in which we added anti-CD40 and anti-CD1d blocking antibodies to CD11bCD15 cells before pulsing them with iNKT cell agonists and incubating them with iNKT cells. Experiments in FIG. 29 were carried out using the same conditions as shown in the right column of FIG. 28 (i.e. in the presence of iNKT cells and alphaGalCer pulsed CD11bCD15 positive cells) in the presence of either blocking anti CD1d (middle column) or blocking anti CD40 (right column) antibodies. The results indicated that the high frequency Melan-A₂₆₋₃₅ specific T cell response (6.42%) was significantly reduced after abolishing the interaction of iNKT cells with CD1d molecules (1.01%) and CD40 (0.86%).

In order to assess whether the cross-talk between CD11bCD15 positive cells may alter the phenotype of CD11bCD15 positive cells, we assessed secretion of ROS by CD11bCd15 positive cells after pulsing them with iNKT cell agonists and co-incubating them with iNKT cells.

FIG. 30 shows that ROS levels in purified MDSC from melanoma patients are reduced by co-incubation with iNKT cells. It can be seen that CD11bCD15 cells from a melanoma patient produce ROS, as defined by the use of the fluorogenic compound DCFDA. After co-incubation with iNKT cells, ROS levels produced by CD11bCD15 cells from a melanoma patient revert to those similar to CD11b+CD15+ cells from a healthy donor. ROS production was assessed in ex-vivo experiments without treating cells with PMA, by incubating cells with the fluorogenic compound DCFDA (right panel) and gating on CD11b CD15 positive cells (left panel). The results of these experiments indicate that the amount of ROS produced by CD11b CD15 positive cells purified from melanoma patients is significantly reduced after co-culture with iNKT cells.

In order to assess whether secretion of IL-10 is contributing to the suppressive activity of CD11bCD15 positive cells, expansion of Melan-A₂₆₋₃₅ specific T cell responses as assessed in the presence or absence of blocking anti 11-10 receptor antibodies. In the left hand panel of FIG. 31, expansion of Melan-A₂₆₋₃₅ specific T cells was assessed using total leukocytes from which CD11b-CD15+ cells were removed (2.33%). Addition of CD11bCD15 positive cells reduced the frequency of Melan-A₂₆₋₃₅ specific T cells (0.83%) (central panel). However, if in the same culture, blocking anti IL-10 receptor antibody was added, the frequency of Melan-A₂₆₋₃₅ specific T cells was significantly enhanced (3.1%) (right hand panel). These results indicate the role of IL-10 secreted by Cd11bCd15 positive cells in suppressing Melan-A₂₆₋₃₅ specific T cell responses.

### Methods

Reagents: Tetramers: UTY₂₄₆₋₂₅₄ H-2 D^{b}, NP₃₆₆₋₃₇₄ H-2 D^{b}, OVA₂₅₇₋₂₆₄ H-2 K^{b} fluorescent tetrameric complexes (tetramers) (J Immunol 171:5140-5147) and CD1d/α-GalCer tetramers (Proc Natl Acad Sci USA 98:3294-3298) were prepared as previously described. α-GalCer was solubilized at 200 μg/ml in ‘vehicle’ (0.5% Tween 20/PBS). Antibodies: The antibodies used for FACS analysis were CD11b, Gr-1-biotin/Streptavidin-, CD86, CD11c, MHC-Class II and TLR9 (all from eBioscience), CD1d, CD40, CD3, and CD8 (BD Bioscience). Dead cells were stained with Bisbenzimide (Sigma-Aldrich) or propidium iodide (Sigma-Aldrich). Samples were acquired on a FACS Calibur™ with CellQuest™ software or on a Cyan Cytometer and analyzed with Flowjo software. The anti CD40 agonist antibody was kindly provided by Prof. Peter Lane (University of Birmingham) (Immunity 5:319-330) and the anti mouse CD1d blocking antibody 3C11 was kindly provided by J. Yewdell (NIH). Viruses: influenza virus strain PR8 (H1N1) was kindly provided by Keith Gould (Imperial College, London) (Virology 95:269-273). Vaccinia virus encoding the UTY₂₄₆₋₂₅₄ was engineered and expanded as previously described (J Immunol 177:983-990). NOS2 and ARG1 inhibitors: N^{G}-monomethyl-L-arginine (LNMMA) (Calbiochem) and N-hydroxy-L-arginine (NOHA) (Calbiochem) were used at 500 μM.

Mice. All mice were maintained in the Biological Services Unit, John Radcliffe Hospital, University of Oxford and used according to established University of Oxford institutional guidelines under the authority of a UK Home Office project license. Female C57BL/6 mice (age 6-8 weeks) were used. CD1d knockout mice (CD1^{-/-}) were kindly provided by Luc Van Kaer (Vanderbilt University School of Medicine, Nashville, Tenn.) (Immunity 6:469-477) and were backcrossed 10 times onto the B6 background, Also used were mice lacking the Jα18 TCR gene segment (Science 278:1623-1626), which were devoid of Vα14 iNKT cells, while having other lymphoid cell lineages intact (iNKT^{-/-} mice). CD40L (CD40^{-/-}) and CD40 (CD40^{-/-}) knock out mice were purchased from The Jackson Laboratory and back-crossed 10 times onto the B6 background (Blood 109:3839-3848). The hexb^{-/-} (Nat Genet. 14:348-352) and iGb3S^{-/-} (Proc Natl Acad Sci USA 104:5977-5982) mice were maintained and genotyped according to published methods. Sandhoff hexb^{-/-} (mouse model of Sandhoff disease) and iGb3S^{-/-} mice had been backcrossed at least six times before use. Heterozygote littermates and age-matched C57BL/6 mice, as appropriate, were used as controls. OT-1 TCR transgenic mice were kindly provided by Dr. M. Merckenschlager (Imperial College London) and back-crossed 8 times onto the B6 background.

In vivo PR8 infection. Female C57BL/6 mice (age 6-8 weeks) were inoculated i.n. with 3×10⁴ pfu of PR8.

Virus Titer. To quantify PR8 virus titres, lungs from PR8 infected mice were harvested 3 days post infection and homogenised. Confluent monolayers of MDCK cells were incubated with 3 fold dilutions of lung homogenates at 37° C. for 30 min. Equal volumes of Type I agarose (1.8% in distilled water, and autoclaved) (Sigma-Aldrich) and MEM containing 2% BSA, 1% glutamine and trypsin (1 μg/ml, Sigma-Aldrich) were heated at 42° C. for at least 45 min before mixing and overlaying on infected cells. Agarose layers were left to set at room temperature. The plates were incubated at 37° C. and plaques counted at day 2. The MDCK cell monolayers were fixed and stained with fixing solution (37% formaldehyde and 1% crystal violet) overnight. The plates were then washed and plaques were counted. Pfu per lung was determined according to the equation: pfu=number of plaques×dilution factor×(total volume of lung homogenate/volume of dilution added to monolayers).

Isolation and culture of mouse MDSC. Purification of MDSC from PR8 infected mice: Lungs from PR8 infected mice (3×10⁴ pfu/mouse) were collected within 4-6 days after the infection and digested with collagenase type 2 (Worthington) for 20 minutes. MDSC were purified from lung homogenates using biotin-conjugated rat anti-mouse Gr-1 antibody and streptavidin-coated magnetic beads following manufacturer's instructions (Miltenyi Biotec). Mouse BM derived MDSC: BM cells were cultured in complete medium with 1 ng/ml GM-CSF. On day 5, CD11c⁺ cells were removed with anti-CD11c-coated magnetic beads (Miltenyi Biotec) and Gr-1⁺ CD11b⁺ CD11c⁻ cells were purified using biotin-conjugated rat anti-mouse Gr-1 antibodies (eBioscience) and streptavidin-coated magnetic beads (Miltenyi Biotec). BM derived MDSC (1×10⁶/well) were treated with 1) α-GalCer (0.4 μg/ml) or 2) TLR ligands (Poly I:C) (Sigma-Aldrich) (0.4 μg/ml), R848 (0.4 μg/ml) (PharmaTech, Shangai, China), CpG 2216 (4 μg/ml) (GGGGGACGATCGTCGGGGGG) (Coley Pharmaceutical), in complete medium for 48 hours or 3) infected with PR8 (2.5×10⁴ pfu) in RPMI at 37° C., 5% CO₂ for 1 h, washed three times and co-cultured with liver purified iNKT cells or OT-I splenocytes.

Identification of human subjects. 89 healthy volunteers were sampled over a 24 month period. Sampling involved taking whole blood PBMC, collection and retaining residual sera/plasma for antibody assays.

Isolation and differentiation of human MDSC. Human MDSC were generated from peripheral blood monocytes. Healthy donor MDSC were differentiated from CD11b⁺ cells by culturing them with 5 ng/ml GM-CSF for 4 days, using a recently described protocol (Immunol Lett 116:41-54.). MDSC purified from IAV infected patients were sorted from PBMC using CD11b beads. α-GalCer and TLR-L were added to human MDSC for 24 hours in the presence of iNKT cells. PR8 (2.5×10⁴ pfu) was added to MDSC in RPMI at 37° C., 5% CO₂ for 1 hour and then cells were washed three times and co-cultured with iNKT cells for 24 h and then irradiated (5000 rad).

Isolation of mouse iNKT cells. Liver iNKT cells were prepared by generating single cell suspensions of perfused livers of naive WT mice. iNKT cells were enriched by overlaying 80% Percoll with cells resuspended in 40% Percoll. Cells were centrifuged at room temperature at 2000 rpm for 25 minutes without brake. Cells were collected from the interphase and iNKT cells were further enriched by sorting with anti-TCRβ and anti-CD5-antibodies (eBioscience), as previously described (Immunity 27:597-609). The % of iNKT cells was measured by CD1d/α-GalCer tetramer staining and ranged from ˜14% to ˜60%. Sorting with anti-TCRβ Ab did not activate iNKT cells, as shown by the lack of IFN-γ and IL-4 secretion by sorted iNKT cells (see FIGS. 4 and 5). In the adoptive transfer experiments 3×10⁵ iNKT cells were transferred i.v. into PR8 infected mice, 24 hours after the infection. iNKT cells were either purified following the protocol described above from WT mice or from Val4 TCR transgenic mice (J Exp Med 188:1831-1839), kindly provided by Agnes Lehuen (INSERM U561, Hôpital Saint Vincent de Paul), using only a Percoll gradient purification, without sorting with anti-TCRβ and anti-CD5-antibodies.

Human iNKT cells. iNKT cells were generated from healthy blood donors as described by Salio et al (Proc Natl Acad Sci USA 104:20490-20495).

Mouse DC differentiation. Mouse DC were differentiated from BM in complete medium in the presence of 20 ng/ml GM-CSF and 20 ng/ml IL-4 (Peprotech) for 7 days. Fresh complete medium, GM-CSF and IL-4 were added every 2 days.

Human DC were generated by culturing monocytes for 4 days in RPMI-10% FCS supplemented with 50 ng/ml GM-CSF (Peprotech) and 1000 U/ml IL-4 (Proc Natl Acad Sci USA 104:20490-20495).

OT-I proliferation assays. Splenocytes from OT-I TCR transgenic mice were pulsed with 2 μg/ml of SIINFEKL peptide for 1 h at 37° C., washed and labelled with 5 μM carboxyluorescein succinimidyl ester (CFSE). Treated or untreated MDSC (3×10⁴) were cultured in 96 well flat bottom plates with 3×10⁵ CFSE labelled OT-I splenocytes. Cells were analyzed using a FACS Calibur™ with CellQuest™ software 4 days later. The data are expressed as the percentage of proliferation of SIINFEKL peptide pulsed and CFSE labelled-OT-I splenocytes, in the presence of MDSC, as compared to proliferation of SIINFEKL peptide pulsed and CFSE labelled-OT-I splenocytes in the absence of MDSC (100%).

Measuring activity of MDSC enzymes. ARG1 and NOS2 activities of α-GalCer pulsed BM-derived MDSC co-cultured with BM derived iNKT cells were assessed at different time points. NOS2 activity was measured from supernatants by Griess reaction, as the amount of NO₃- and NO₂-produced using a nitrate/nitrite assay kit (Cayman Chemicals) (Proc Natl Acad Sci USA 102:4185-4190). ARG1 activity was measured in cell lysates, as previously described (Proc Natl Acad Sci USA 102:4185-4190).

Prime-boost vaccination. MDSC (5×10⁶) purified from female mice were injected i.v. into female recipients. On the same day, BM derived DC (1×10⁶) from male mice, were injected i.v. in the opposite tail vein. A week later, mice were boosted with 10⁶ pfu of UV-inactivated vaccinia virus encoding the Uty₂₄₆₋₂₅₄ peptide (J Immunol 177:983-990).

Anti IAV antibody measurements: Plasma taken from individuals was stored at −80° C. prior to assay for the presence of Haemagglutination inhibiting (HI) antibodies. Samples were treated A Wellington/1/2004 A Wellington/1/2004 with receptor-destroying enzyme for 16 hours at 37° C. (13v/v), followed by inactivation at 56° C. (RDE, Denka Seken Co Ltd., Japan). HI assays were performed in duplicate using standard protocols (Laboratory Diagnosis of Influenza. Textbook of Influenza Blackwell 291-313) with whole virus antigens representing influenza A viruses circulating during the winter seasons of 2006-7 and 2007-8. A/Solomon Islands/3/2006 and A/New Calcdonia/20/99 (H1N1 strains) and A/Wisconsin/67/2005 and A Wellington/1/2004 (H3N2 strains) circulating during the time period over which the human material was collected. The HI antibody titer was calculated as the highest dilution that completely prevented agglutination. The final titer was the geometric mean of the duplicates. Sequential sera from the same individual were assayed simultaneously. Fourfold or greater rises in HI antibody titer between pre-infection and post illness sera were considered indicative of recent infection.

Mixed Lymphocyte Reaction (MLR). Peripheral blood lymphocytes (PBL) (2×10⁵) were mixed with allogeneic irradiated (5000 rad) DC (5×10⁴) in 200 μl of RPMI 5% human AB serum in 96-well flat-bottom plates. Cells were incubated at 37° C., 5% CO₂ for 5 days and then 1 μCi/well ³H-thymidine (Perkin Elmer life Sciences, Boston, Mass.) was added for 15-18 h. ³H-thymidine incorporation was measured using a Wallac Microbeta Jet 1450 reader (Perkin Elmer). MDSC-mediated inhibition of lymphocyte proliferation was carried out by co-culturing irradiated MDSC, (5×10⁴), from either healthy donors or influenza virus infected patients, together with BL and irradiated DC. The data are expressed as the percentage of PBL proliferation driven by allogeneic irradiated DC in the presence of irradiated MDSC, as compared to alloreactive PBL proliferation in the absence of MDSC (100%).

ELISA: 1) Cytokines. Supernatants of MDSC co-cultured with BM derived iNKT cells were collected at different time points. The amount of IL-12p40, IFN-γ and IL4 was measured using an ELISA kit (eBioscience). 2) IgG Antibodies. Sera from PR8 infected mice were collected 6 days post infection and antibody titers were determined by coating 96-well maxisorb NUNC plates with 5 ng/ml of PR8 virus overnight at 4° C. The plates were washed with PBS and blocked with 1% of BSA for 1 h at room temperature. Serial dilutions of the serum samples were plated for 2 h at room temperature. Plates were then washed, and horse radish peroxidase-coupled goat anti-mouse IgG was added for 1 h. Color reactions were developed with 3,3′,5-5′-Tetramethylbenzidine (TMB) (Sigma-Aldrich) and the absorbance was measured at 490 nm.

RT-PCR. Human and mouse MDSC that were infected in vitro or in vivo were checked for infection by RT-PCR using the following NP primers:

Forward-TGATCGGAACTTCTGGAGGG
and
Reverse-TGGCCCAGTACCTGCTTCTC.

As control Glyceraldehyde 3-phosphate dehydrogenase (mouse GAPDH):

Forward-CCAGGTTGTCTGCGACTT
and
Reverse-CCTGTTGCTGTAGCCGTATTC.

Human GAPDH-RP:

Forward-CCAGCCGAGCCACATCGCTC
and
Reverse-ATGAGCCCCAGCCTTCTCCAT.

Purification of CD11b/CD15+ cells. Since CD15 CD11b positive cells have granulocyte density, it is important to purify them without using density gradients, such as Ficol. Red blood cells are lysed from whole blood, using red lysis cell buffer and anti CD11b/CD15 specific antibodies are added to the leukocytes for FACS guided cell sorting.

Melan-A priming experiments. DCs were generated by culturing monocytes for 4 days in RPMI/10% FCS supplemented with 50 ng/ml GM-CSF (Pepro-tech) and 1,000 U/ml IL-4 (Salio, M., et al. 2007. Proc. Natl. Acad. Sci. U.S.A. 104:20490-20495). Total leukocytes or Ficoll purified PBL (which do not contain CD11bCD15 positive cells) were then added for 2 weeks to melan-A26-35 pulsed DC. After 2 weeks, cells were stained with HLA-A2 tetramers loaded with melan-A26-35 peptide and anti CD8.

Harnessing iNKT cells. The protocol to purify Cdl lbCd15 cells and differentiate DC is similar to the protocol described earlier. Cdl lbCd15 cells were pulsed with either alphaGalCer (100 ng) or with threitolceramide (200 ng) and then incubated with iNKT cells for 12 hours. Cells were then washed pulsed with the melan-A26-35 peptide and incubated with total leukocytes. After 2 weeks in culture, cells were stained with HLA-A2 melan-A tetramers and anti-CD8 antibodies.

1. A method of reducing myeloid-derived suppressor cell activity (MDSC) in a mammalian subject comprising administering to the subject a pharmaceutically acceptable amount of an iNKT agonist, a TLR agonist, or combination thereof, wherein the subject has elevated MDSC activity prior to administration.

2. A method according to claim 1, wherein the mammalian subject is elderly or is subject to extreme stresses or is suffering from cancer.

3. A method according to claim 1, wherein the elevated MDSC activity is due to a disease.

4. A method according to claim 3, wherein the disease is selected from influenza A virus (IAV), pandemic flu, vaccinia virus infection, polymicrobial sepsis, chronic microbial infection, severe trauma, parasitic infections or cancer.

5. A method of stimulating an immune response in a mammalian subject against a disease comprising administering to the subject (i) a pharmaceutically acceptable amount of an iNKT agonist, a TLR agonist, or combination thereof, and subsequently administering to the subject (ii) an immunotherapeutic composition.

6. A method according to claim 5 wherein the immunotherapeutic composition comprises a vaccine, and the vaccine is administered 1 to 28 days after the treatment of (i).

7. A method according to claim 5 wherein the disease is selected from influenza A virus (IAV), pandemic flu, vaccinia virus infection, polymicrobial sepsis, chronic microbial infection, severe trauma, parasitic infections cancer, hepatitis B virus, hepatitis C virus or pandemic flu.

8. A method of treatment or prophylaxis for an acute infectious disease which induces a high level of myeloid-derived suppressor cells (MDSCs) in a subject suffering from the acute infection disease, the method comprising administering to the subject a pharmaceutically acceptable amount of an iNKT agonist, a TLR agonist, or combination thereof.

9. A method according to claim 8 wherein the disease is influenza A virus.

10. A method according to claim 8, wherein the subject does not have the acute infection disease and the method is a prophylactic treatment.

11. (canceled)

12. A method according to claim 10 wherein the disease is influenza A virus (IAV), pandemic flu, vaccinia virus infection polymicrobial sepsis, chronic microbial infection, severe trauma, parasitic infections cancer, hepatitis B virus, hepatitis C virus or pandemic flu.

13. A method according to claim 1 wherein the iNKR agonist is an α-GalCer analogue.

14. A method according to claim 13 wherein the α-GalCer analogue is threitolceramide:

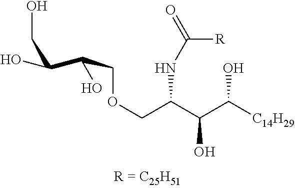

15. A method according to claim 1 wherein the TLR agonist is selected from poly I:C (TLR3), MPL (TLR4), imiquimod (TLR7), R848 (TLR7/8), 8852 (TLR7), R853 (TLR8), R34240 (TLR7), R854 (TLR7/8), CpG (TLR9), or combination thereof.

16.-30. (canceled)
